(19) United States
(12) Patent Application Publication
(10) Pub. No.: US 2024/0197222 A1
(43) Pub. Date: Jun. 20, 2024

(54) ORAL CARE FLAVOUR FOR IMPROVING HAPPINESS STATE AND METHOD OF ASSESSING

(71) Applicant: GIVAUDAN SA, VERNIER (CH)

(72) Inventors: Giuliano GAETA, Ramsgate Kent (GB); Alan Forbes PROVAN, Kingsnorth, Ashford Kent (GB); Imogen Maria MATHEWS, Richmond, Greater London (GB); Salla Katja Emilia PARKKINEN, Kajaani (FI)

(21) Appl. No.: 18/555,838

(22) PCT Filed: May 30, 2022
(86) PCT No.: PCT/EP2022/064557

§ 371 Date: Oct. 17, 2023

(30) Foreign Application Priority Data
May 28, 2021 (GB) 2107716.9

(51) Int. Cl. A61B 5/16; A61B 5/00

(52) U.S. Cl. CPC A61B 5/165; A61B 5/0075

(57) ABSTRACT

The present disclosure relates to methods of assessing the ability of a test oral care flavour ingredient or a test oral care flavour composition to improve the happiness state of a human subject and of creating oral care flavour compositions having a positive happy effect on a human subject. It further relates to oral care flavour compositions for improving the happiness state of a human subject, to consumer products comprising such oral care flavour compositions, and to methods of improving the happiness state of a human subject.

The present invention relates to methods of assessing the ability of a test oral care flavour ingredient or a test oral care flavour composition to improve the happiness state of a human subject and of creating oral care flavour compositions having an effect of improving the happiness of a human subject. It further relates to oral care flavour compositions for improving the happiness state of a human subject, to consumer products comprising such oral care flavour compositions, and to methods of improving the happiness state of a human subject.

Fragrances and flavours have been widely employed by consumer product companies in order to impart to their products pleasant, well-liked odours and tastes that promote consumer liking and which influence purchasing decisions for this reason. However, in an increasingly competitive market place, mere liking is often not sufficient to differentiate one brand over its competitors. Accordingly, in the market execution of their products, consumer product companies frequently refer to wide-ranging product benefits, typically communicated through diverse advertising campaigns, as well as on the packaging and labelling of their products, which together form an important part of their branding strategy. New differentiating effects are constantly sought, and flavours have often been employed as a means to achieve those effects. For example, flavours have been employed to create real or perceived functional effects that may relate to oral care, such as hygiene effects, malodour-counteracting effects, and the like.

In addition to its functional effects, odour and taste are known to elicit emotional responses in human subjects. The temporary beneficial psychological effects of odour on human emotions have been studied extensively in the academic literature. For example, the effects of odour on the affective (i.e. emotional) experience of human subjects have been studied by C. Chrea et al. (2009), “Mapping the semantic space for the subjective experience of emotional responses to odours”, Chemical Senses, 34 (2009) 49-62 and S. Delplanque et al. “How to map the affective semantic space of Scents”, Cognition & Emotion 26(5) (2012) 885-898. In particular, these papers describe a verbal measurement method in the form of a questionnaire, wherein the measurement terms are selected such that it is possible to differentiate the various moods and emotions evoked by odours emanating from perfumed and flavoured products. However, in these papers, the cognitive aspect of well-being is under-estimated and limited to memory aspects associated with the olfactory function.

It has long been known that fragrance materials and essential oils can promote feelings of happiness and well-being. These materials have also been used in cosmetic products and aromatherapy in order to provide a similar effect.

Aroma-Chology® is a term coined by the Olfactory Research Fund Ltd. (see extensive review by J. Jellinek in Cosmetics & Toiletries, (1994) 109, pp 83-101). It is concerned specifically with the temporary, beneficial psychological effects of aromas in human behaviours and emotions to improve mood and quality of life. In fact, a large number of products promoted as having aromatherapy benefits can be more accurately identified for their Aroma-Chology® benefits as they produce temporary psychological effects. However, there is no teaching as to how to formulate products to achieve such benefits qualitatively or quantitatively with a reliable expectation of success. In addition, it is well known that fragrances and flavours can be perceived as associated with different attributes in different countries.

More recently, several patent applications (e.g. WO 02/49600, WO 2008/050084, WO 2008/050086, WO 2020/165464) have focused on providing positive mood benefits through fragrance and flavour compositions, providing guidelines on how to measure these mood benefits and on how to create effective fragrance and flavour compositions.

For instance, WO 2020/165464 describes oral care flavour compositions that enhance well-being, as well as a method of measuring the impact of oral care flavour compositions on the well-being of human subjects. This method employs a questionnaire that breaks-down the concept of well-being into key attributes that have an effect on affective or cognitive elements of well-being. The key attributes are weighted in a manner that reflects the relative importance of each attribute to the holistic concept of well-being.

Thus, WO 2020/165464 provides a method for assessing the overall effect on well-being. It would be interesting, though, to be able to assess the effect of a fragrance or flavour composition on specific moods and emotions, such as relaxation, invigoration, or happiness, for instance.

Furthermore, WO 2020/165464 uses consumer testing for assessing the influence of an oral care flavour composition on the mood and emotions of test subjects. However, consumer testing has several important drawbacks:
Lack of any physiological information from the participant
No information on implicit mental processes happening in the brain
Explicit/declarative description of odours is much harder than other stimuli (e.g. the description of a picture), often resulting in inaccurate findings
Participants are able to control the answers provided to a test and might alter their answers to please the experimenter (a phenomenon known in the psychology literature as “participant bias”), leading to the collection of inaccurate data (https://doi.org/10.1016/0191-8869(86)90014-0)
Difficulty to differentiate different mood states between samples
Ratings often correlate with liking scores
Tendency to acquiesce—answer positively
Tendency to be evasive—to opt for neutral, unsure, uncertain response
Tendency for speed—to complete the test quickly rather than accurately It is therefore highly desirable that an improved technique for assessing the mood state of a human subject is developed, which allows for more flexibility during the measurement.

The above problems are solved by the present invention.

In a first aspect, the present invention provides a method of assessing the happiness state of a human subject by means of fNIRS (functional near-infrared spectroscopy).

In a second aspect, the present invention provides a method of assessing the ability of a test oral care flavour ingredient or a test oral care flavour composition to improve the happiness state of a human subject.

In a third aspect, the present invention provides a method of creating an oral care flavour composition having an effect of improving the happiness of a human subject.

In a fourth aspect, the present invention provides an oral care flavour composition for improving the happiness state of a human subject.

In a fifth aspect, the present invention provides a consumer product comprising said oral care flavour composition.

In a sixth aspect, the present invention provides a method of improving the happiness state of a human subject, comprising the step of providing an effective amount of the oral care flavour composition of the invention to the human subject.

In a seventh aspect, the present invention relates to the use of certain oral care flavour ingredients for improving the happiness state of a human subject.

The use of functional near-infrared spectroscopy (fNIRS) for assessing the mood state, and in particular the happiness state, of a human subject is highly advantageous: fNIRS is harmless, tolerant to bodily movements, and highly portable; it is also suitable for all possible participant populations, from newborns to the elderly, and experimental settings, both inside and outside the laboratory (for a review, see: “The present and future use of functional near-infrared spectroscopy (fNIRS) for cognitive neuroscience”, Pinti et al., Ann. N.Y. Acad. Sci. 1464 (2020) 5-29). In particular, the use of fNIRS allows for in-context testing, where participants are asked to perform specific tasks related to the fragrances provided to them (e.g. cleaning a hard surface while smelling the fragrance of an all-purpose cleaner).

fNIRS is an optical, non-invasive neuroimaging technique that allows the measurement of brain tissue concentration changes of oxygenated (Oxy Hb or HbO₂) and deoxygenated (Deoxy Hb or HbR) haemoglobin following neuronal activation. This is achieved by shining NIR light (650-950 nm) into the head, and, taking advantage of the relative transparency of the biological tissue within this NIR optical window, light will reach the brain tissue. The most dominant and physiological-dependent absorbing chromophore within the NIR optical window is haemoglobin. Based on its saturation state, we can have haemoglobin in its oxygenated (HbO₂) and deoxygenated form (HbR). In particular, HbO₂ and HbR absorb the NIR light differently: HbO₂ absorption is higher for λ>800 nm; on the contrary, HbR absorption coefficient is higher for λ<800 nm.

When a brain area is active and involved in the execution of a certain task, the brain's metabolic demand for oxygen and glucose increases, leading to an oversupply in regional cerebral blood flow (CBF) to meet the increased metabolic demand of the brain. The oversupply in regional CBF produces an increase in HbO₂ and a decrease in HbR concentrations; these are estimated by changes in light attenuation that can be measured by fNIRS.

The portion of tissue interrogated by the NIR light is called a channel and is located at the midpoint between the source optode (S) and the detector optode (D), and at a depth of around the half of the source-detector separation. To fully exploit the potential of fNIRS, multi-channel devices are used nowadays. These allow monitoring of larger portions of the head and the gathering of topographic HbO₂ and HbR maps. Several multi-channel fNIRS devices are commercially available (e.g. Brite by Artinis, ETG-4100 by Hitachi or NIRSPort by NIRx).

The position of the fNIRS channels is generally standardized based on the EEG's 10-20 system. Typical devices use about sixteen to twenty-two channels. Optodes (detectors and sources) must be placed in an alternate fashion (i.e. a source followed by a detector, followed by a source . . . ) typically in a grid with equal distances between the channels, e.g. at a distance of 3 cm (Pinti et al. (2019) “Current Status and Issues Regarding Pre-processing of fNIRS Neuroimaging Data: An Investigation of Diverse Signal Filtering Methods Within a General Linear Model Framework”, Front. Hum. Neurosci. 12:505.). Both optodes and channels are typically numbered to allow for identification. For optodes, the letter S before the number typically defines a source optode, while the D letter before the number defines a detector optode. The numbers are usually progressive, e.g. from 1 to 8 for the sources and from 1 to 7 for the detectors.

In the methods of the present invention, the following set-up of the fNIRS channels was used:

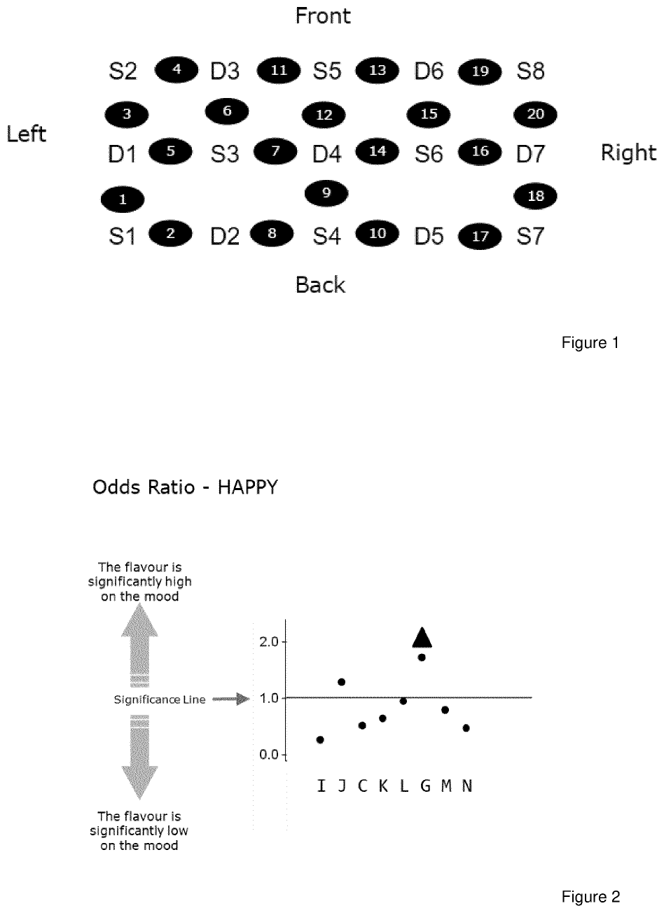

The centre of fNIRS Channel 12 was placed on the standard position EEG channel FPz according to the EEG 10-20 system (Trambaiolli et al. “Predicting affective valence using cortical hemodynamic signals”, Sci Rep 8, 5406 (2018)). Channel 12 is located between source S5 and detector D4, where S5 is situated 1.5 cm from the location of the EEG channel FPz towards the Nasion on the midline of the head, and D4 is situated 1.5 cm from the location of EEG channel FPz towards the Inion on the midline of the head. All fNIRS optodes are placed at a standardised distance of 3 cm one from one another and are arranged on gridlines extending parallel and orthogonally to the midline. Taking S5 and D4 as a reference, and considering a shift of 3 cm for each optode either on the Nasion-Inion direction (where “in front” means towards the Nasion and “behind” means towards the Inion) or on the Pre Auricolar line (where “to the left” means towards the Left Pre Auricolar line and “to the right” means towards the Right Pre Auricolar Line), then S4 is behind D4, D5 on the right of S4, S7 on the right of D5, D7 in front of S7, S8 in front of D7, S6 on the left of D7, D6 on the left of S8, D2 on the left of S4, S3 on the left of D4, D3 in front of S3, S1 on the left of D2, D1 in front of 51 and S2 in front of D1. This setup is also shown in FIG. 1.

The channel scheme is the following:

| Channel | Position |
|---|---|
| Number | (Source - Detector) |
| Channel 1 | S1 - D1 |
| Channel 2 | S1 - D2 |
| Channel 3 | S2 - D1 |
| Channel 4 | S2 - D3 |
| Channel 5 | S3 - D1 |
| Channel 6 | S3 - D3 |
| Channel 7 | S3 - D4 |
| Channel 8 | S4 - D2 |
| Channel 9 | S4 - D4 |
| Channel 10 | S4 - D5 |
| Channel 11 | S5 - D3 |
| Channel 12 | S5 - D4 |
| Channel 13 | S5 - D6 |
| Channel 14 | S6 - D4 |
| Channel 15 | S6 - D6 |
| Channel 16 | S6 - D7 |
| Channel 17 | S7 - D5 |
| Channel 18 | S7 - D7 |
| Channel 19 | S8 - D6 |
| Channel 20 | S8 - D7 |

Thus, there are nine channels per hemisphere (left or right) and two channels at the midline of the frontal and prefrontal areas. Channels 1 to 8 and 11 are located in the left hemisphere, Channels 9 and 12 are on the midline, and Channels 10 and 13 to 20 are in the right hemisphere. Channels 9 and 12 are only considered for full brain analysis.

By means of extensive research, it has been found that certain areas of the brain, and in particular certain channels, can be used as indicators for assessing the happiness state of a human subject. More specifically, an increase or decrease of Oxy Hb, Deoxy Hb and/or Total Hb (corresponding to the sum of Oxy Hb plus Deoxy Hb) in the left or right hemisphere, the full brain, or certain specific channels, at certain time points provides an indication as to whether the happiness state of the human subject is increased or decreased or stays about the same. The details will be described in relation to the method outlined below, but equally apply to the general method of assessing the happiness state of a human subject.

The above finding has been applied in the present invention to provide a method of assessing the ability of a test oral care flavour ingredient or a test oral care flavour composition to improve the happiness state of a human subject.

Said method comprises the following steps:
1. measuring a base happiness state of one or more human test subject(s);
2. providing the test oral care flavour ingredient or the test oral care flavour composition to the human test subject(s) for oral application;
3. measuring a resulting happiness state of the human test subject(s); and
4. determining a difference between the resulting happiness state and the base happiness state for the human test subject(s).

The base happiness state and the resulting happiness state are measured by functional Near Infrared Spectroscopy (fNIRS) of the human test subject(s)' left brain hemisphere, right brain hemisphere, and full brain.

In order to measure the base happiness state, the human test subject(s) may be provided with a non-flavoured sample, e.g. water.

Alternatively, it is also possible to measure a reference happiness state, e.g. in the presence of a reference mouthwash or toothpaste sample.

The samples are provided for oral application. Throughout this disclosure, “oral application” is meant to encompass all means of orally applying a material without swallowing it, including, but not limited to, gargling with a liquid, spraying a liquid or aerosol, brushing one's teeth with a gel or paste, chewing a chewing gum, rubbing the material onto the gums or the lips, using a dental floss, chewing a toothpaste or mouthwash tablet, applying a whitening film on the teeth and/or gums, applying a denture fixative gel or cream, and otherwise applying the material to the gums, tongue, palate, teeth, lips and/or throat.

It has been found that the duration of the oral application is not relevant for the results.

If more than one human test subjects are involved, results for the base happiness state and the resulting happiness state may be averaged prior to determining the difference. Alternatively, it is also possible to determine the difference for each human test subject separately.

It has been found that the test oral care flavour ingredient or the test oral care flavour composition is able to improve the happiness state of the human subject if at least one out of the following eight conditions A1 through A8 is met:
A1. Channel 14 shows a statistically significant difference of Total Hb 0-10 seconds after the oral application;
A2. Channel 15 shows a statistically significant difference of Total Hb 0-10 seconds after the oral application;
A3. Channel 18 shows a statistically significant difference of Total Hb 0-10 seconds after the oral application;
A4. Channel 19 shows a statistically significant difference of Total Hb 0-10 seconds after the oral application;
A5. Channel 1 shows a statistically significant difference of Total Hb 0-10 seconds after the oral application;
A6. Channel 3 shows a statistically significant difference of Total Hb 0-10 seconds after the oral application;
A7. Channel 4 shows a statistically significant difference of Total Hb 0-10 seconds after the oral application;
A8. Channel 5 shows a statistically significant difference of Total Hb 0-10 seconds after the oral application.

As outlined above, Total Hb is the amount of total haemoglobin measured, Oxy Hb is the amount of oxygenated haemoglobin measured, and Deoxy Hb is the amount of deoxygenated haemoglobin measured.

Haemoglobin values for the left brain hemisphere correspond to the mathematical average of the individual haemoglobin values of Channels 1 to 8 and 11, as defined above.

Haemoglobin values for the right brain hemisphere correspond to the mathematical average of the individual haemoglobin values of Channels 10 and 13 to 20, as defined above.

Haemoglobin values for the full brain correspond to the mathematical average of the individual haemoglobin values of all Channels 1 to 20, as defined above.

The effect on haemoglobin levels (Total Hb, Oxy Hb, and Deoxy Hb) may vary over time. It was found that more accurate results can be obtained by analysing haemoglobin values for several different time periods, e.g. after 0-5 seconds, 0-10 seconds, 5-10 seconds, 10-15 seconds, 15-20 seconds, 10-20 seconds, 20-25 seconds, 25-30 seconds, or after 30 seconds. These time windows are optimal choices to evaluate changes in fNIRS signals as they provide enough time to the HRF (Hemodynamic Response Function) to fluctuate (peak time is around 3-5 seconds after the initial presentation of a stimulus) and display differences between two conditions.

Statistical significance is verified using a 2-tailed Student's t-test with a statistical significance threshold at 0.05.

Throughout this application, the terms “improving the happiness state”, “increasing the happiness state”, and “enhancing the happiness state”, and “increasing happiness” are used interchangeably. They are meant to express that a certain item, in particular an oral care flavour ingredient or oral care flavour composition or consumer product containing the same, has a positive mood enhancing effect on a human subject. In other words, they induce positive moods and emotions, such as happiness, and make people feel happy, uplifted, delighted, optimistic, and enthusiastic.

This emotional territory has been defined typically by the model circumplex of affect (Posner J, Russell J A, Peterson B S. The circumplex model of affect: an integrative approach to affective neuroscience, cognitive development, and psychopathology. Dev Psychopathol. 2005; 17(3): 715-734. doi:10.1017/S0954579405050340), with happy positive moods represented by feelings of happiness, excitement and contentedness.

This emotional space has now been found to further include positive emotions, such as optimism and fulfilment, to reflect those inner feelings of happiness, and also highly activated emotions, such as delight, enthusiasm, euphoria and playfulness often shared with others.

Thus, the present application in general relates to the enhancement of positive emotions spanning the range of happiness: from positive low activated mood states, such as contentedness, to highly activated positive happy moods, such as excitement. These positive mood states of happiness include, but are not limited to, those inner feelings of happiness, such as optimism and fulfilment, and those highly activated feelings of happiness, such as delight, enthusiasm, euphoria and playfulness.

Throughout this application, the terms “flavour”, “taste” and “aroma” are used interchangeably.

Furthermore, also the terms “(oral care flavour) ingredient” and “(oral care flavour) material” are used interchangeably. In the context of the present invention, the term “oral care flavour ingredient” refers to an ingredient that has the function of providing a noticeable and identifiable flavour to the oral care composition. Oral care flavour ingredients include highly performing ingredients intended for providing an intense impression, as well as less performing ingredients intended for providing a subtle impression.

The term “flavour composition” relates to a mixture of two or more flavour ingredients. It may optionally include one or more tasteless or low-taste solvents and/or diluents, e.g. as a vehicle for a flavour material.

Throughout this application, the terms “(human) test subjects” and “participants” are used interchangeably.

Preferably, several human test subjects are involved in the method of the invention, in order to get a more representative and reliable result, for example more than five, more than ten, more than 15, or even more. Results from several human test subjects may be averaged. Alternatively, they may also be summed up.

Furthermore, participants that indicate that they dislike a certain test oral care flavour ingredient or test oral care flavour composition may be excluded from the respective analysis.

The method of the present invention allows for a fast, simple and reliable assessment of the ability of a test oral care flavour ingredient or a test oral care flavour composition to improve the happiness state of a human subject. Oral care flavours may be tested in wide variety of settings, from a non-motion laboratory setting to in-context-testing. Furthermore, the method allows for detecting sub-conscious effects, thereby avoiding common issues of conscious methods (e.g. interrogation), which often provide only limited and often inaccurate information due to dishonest responses, prior survey biases, and inarticulacy, for instance.

In order to qualify as a happy oral care flavour ingredient or oral care flavour composition, a test oral care flavour ingredient/composition must fulfil at least one of the eight conditions A1 through A8. Preferably, at least two out of the conditions A1 through A8 are met, more preferably at least three, and most preferably at least four.

Most reliable results were observed for conditions A2, A3, A4, A5, A6, A7, and A8.

Therefore, in an embodiment, at least one, preferably at least two, of the following conditions are met: A2, A3, A4, A5, A6, A7, and A8.

More specifically, the applicant has identified certain specific Channels, haemoglobin types and time points that are particularly indicative of the effect on the happiness state of the human subject.

Therefore, in an embodiment, at least one, more preferably at least two, and most preferably at least three, out of the following nine conditions 1 through B9 is met:
1. Channel 14 shows a statistically significant difference of Oxy Hb 0-10 seconds after the oral application;
B2. Channel 16 shows a statistically significant difference of Oxy Hb 0-10 seconds after the oral application;
B3. Channel 18 shows a statistically significant difference of Oxy Hb 0-10 seconds after the oral application;
B4. Channel 20 shows a statistically significant difference of Oxy Hb 0-10 seconds after the oral application;
B5. Channel 2 shows a statistically significant difference of Oxy Hb 0-10 seconds after the oral application;
B6. Channel 3 shows a statistically significant difference of Oxy Hb 0-10 seconds after the oral application;
B7. Channel 4 shows a statistically significant difference of Oxy Hb 0-10 seconds after the oral application;
B8. Channel 5 shows a statistically significant difference of Oxy Hb 0-10 seconds after the oral application;
B9. Channel 6 shows a statistically significant difference of Oxy Hb 0-10 seconds after the oral application.

Most reliable results were observed for conditions B2, B3, B4, B5, B7, and B8.

Therefore, in an embodiment, at least one, preferably at least two, of the following conditions are met: B2, B3, B4, B5, B7, and B8.

It was found that the efficacy of the oral care flavour ingredient or composition for providing a happy effect could be even further improved if at least one, more preferably at least two, and most preferable at least three, out of the following eight conditions C1 through C8 is met:
C1. Channel 16 shows a statistically significant difference of Total Hb 5-10 seconds after the oral application;
C2. Channel 19 shows a statistically significant difference of Total Hb 5-10 seconds after the oral application;
C3. Channel 20 shows a statistically significant difference of Total Hb 5-10 seconds after the oral application;
C4. Channel 1 shows a statistically significant difference of Total Hb 5-10 seconds after the oral application;
C5. Channel 4 shows a statistically significant difference of Total Hb 5-10 seconds after the oral application;
C6. Channel 6 shows a statistically significant difference of Total Hb 5-10 seconds after the oral application;
C7. Channel 9 shows a statistically significant difference of Total Hb 5-10 seconds after the oral application;
C8. Channel 12 shows a statistically significant difference of Total Hb 5-10 seconds after the oral application.

Most reliable results were observed for conditions C1, C4, C5, C6, and C8.

Therefore, in an embodiment, at least one, preferably at least two, of the following conditions are met: C1, C4, C5, C6, and C8.

Based on the above described method of assessment, it was possible to develop guidelines for creating oral care flavour compositions that have an effect of improving the happiness of human subjects.

Therefore, the present invention also provides a method of creating an oral care flavour composition having an effect of improving the happiness of a human subject, comprising the steps of:
(i) creating a test oral care flavour composition;
(ii) assessing the ability of the test oral care flavour composition to improve the happiness state of a human subject according to the method described above; and
(iii) adjusting, if necessary, the test oral care flavour composition by adding and/or removing at least one oral care flavour ingredient and/or increasing and/or reducing the concentration of at least one oral care flavour ingredient until the oral care flavour composition is found to improve the happiness state of the human subject.

Therefore, it is first assessed whether or not the test oral care flavour composition provides an effect of increasing happiness. Subsequently, if necessary, the composition is adjusted to create an improved oral care flavour composition.

Steps (ii) and (iii) may be repeated if necessary and/or desired.

Increasing the level of happy ingredients, such as HMP, HMR, and HMI, increases the likelihood that the oral care flavour composition would have a suitable character to deliver the happiness benefit. Other ingredients reduce the likelihood that the benefit will be achieved, as their level in the oral care flavour composition is increased, e.g. INV or REL.

Therefore, in one embodiment, at least one HMP oral care flavour ingredient is added to the (test) oral care flavour composition in step (iii).

Alternatively or in addition, at least one HMR oral care flavour ingredient may be added to the (test) oral care flavour composition in step (iii).

Alternatively or in addition, at least one HMI oral care flavour ingredient may be added to the (test) oral care flavour composition in step (iii).

Alternatively or in addition, at least one INV oral care flavour ingredient may be removed from the (test) oral care flavour composition in step (iii).

Alternatively or in addition, at least one REL oral care flavour ingredient may be removed from the (test) oral care flavour composition in step (iii).

Alternatively or in addition, the concentration of at least one HMP oral care flavour ingredient may be increased in step (iii).

Alternatively or in addition, the concentration of at least one HMR oral care flavour ingredient may be increased in step (iii).

Alternatively or in addition, the concentration of at least one HMI oral care flavour ingredient may be increased in step (iii).

Alternatively or in addition, the concentration of at least one INV oral care flavour ingredient may be reduced in step (iii).

Alternatively or in addition, the concentration of at least one REL oral care flavour ingredient may be reduced in step (iii).

It has been found that the following oral care flavour ingredients have a happy effect and are, thus, HMP oral care flavour ingredients: fruity-strawberry ingredients, fruity-raspberry ingredients, fruity-apple ingredients, fruity-banana ingredients, floral-freesia ingredients, floral-lily of the valley ingredients, animalic-butyric ingredients, acetic acid, and mixtures thereof.

It has been found that the following oral care flavour ingredients have a happy-relaxing effect and are, thus, HMR oral care flavour ingredients: fruity-pineapple ingredients, fruity-peach ingredients, herbal-spearmint ingredients, lemon oil, lemon oil reconstitutions, and mixtures thereof.

It has been found that the following oral care flavour ingredients have a happy-invigorating effect and are, thus, HMI oral care flavour ingredients: citrus-orange ingredients, spicy-cinnamon ingredients, floral-jasmine ingredients, cooling ingredients, aromatic-wintergreen ingredients, lime terpenes, lime terpeneless, and mixtures thereof.

It has been found that the following oral care flavour ingredients have an invigorating effect and are, thus, INV oral care flavour ingredients: citrus-lime ingredients (excluding lime terpenes and lime terpeneless), citrus-mandarin ingredients, citrus-grapefruit ingredients, spicy-pepper ingredients, spicy-clove ingredients, herbal-rosemary ingredients, green-grass ingredients, woody-resinous ingredients, herbal-coniferous ingredients, bergamot oils, 3,7-dimethylocta-1,6-dien-3-yl acetate (linalyl acetate), 3,7-dimethylocta-1,6-dien-3-yl 2-methylpropanoate (linalyl iso butyrate), (E)-3,7-dimethylocta-2,6-dienal (citral), lemongrass, Litsea cubeba oils, and mixtures thereof.

It has been found that the following oral care flavour ingredients have a relaxing effect and are, thus, REL oral care flavour ingredients: sweet-vanilla ingredients, sweet-coconut ingredients, sweet-cooked sugar ingredients, fruity-passionfruit ingredients, herbal-thyme ingredients, sage oils and reconstitutions, clary sage oils and reconstitutions, camomile oils and reconstitutions, lavender oils and reconstitutions, and mixtures thereof.

Where trivial names are used to describe useful flavour ingredients herein, the skilled flavourist will understand that these are commonly used names in the art of flavour design. However, the skilled flavourist would also understand that these ingredients may also be known by other trivial synonyms, by CAS registry numbers, or by more formal nomenclature, such as IUPAC nomenclature. Furthermore, the skilled flavourist would be familiar with these other trivial synonyms, as well as with more formal nomenclature, or at the least, would be aware of standard reference works, such as The Good Scents Company website, which contains a comprehensive list of trivial names, registry numbers and more formal nomenclature for the flavour ingredients contained in the flavourists' palette.

Flavour compositions and individual flavour ingredients may be characterized by their flavour attributes. Although flavour creation is part science and part artistry, and there is no absolute prescribed definition for flavour attributes of flavour compositions and flavour ingredients, nevertheless trained flavourists, realizing that there will be margin for some subjectivity, will be able to assign flavour compositions and ingredients to a general flavour descriptor and a flavour family.

Flavour families provide a general description of a flavour space, and their number is usually limited. Hence, most of the ingredients used in flavour creation and particularly useful in the context of the present invention may be described by a small set of flavour families selected from the group consisting of “aldehydic”, “ambery”, “animalic”, “aromatic/herbal”, “citrus”, “cooling”, “earthy”, “floral”, “fruity”, “green”, “musky”, “roasted”, “spicy”, “sweet”, “warming”, “watery”, and “woody”.

Flavour descriptors provide a more accurate description of the flavour of a flavour composition or ingredient within a family. They are more abundant and their number and diversity is often unlimited. Examples of flavour descriptors include, but are not limited to, “almond”, “ambergris”, “anis”, “apple”, “armoise”, “balsam”, “banana”, “blackcurrant”, “butter”, “butryric”, “camomille”, “candied fruit”, “caramel”, “cardamom”, “caraway”, “cinnamon”, “citronella”, “clove”, “cocoa”, “coconut”, “coffee”, “coniferous”, “cooked sugar”, “coriander”, “cream”, “cucumber”, “cumin”, “eucalyptus”, “freesia”, “ginger”, “grapefruit”, “grass”, “heliotrope”, “honey”, “jasmine”, “lavender”, “leaf”, “lemon”, “lily of the valley”, “lime”, “liquor”, “mandarin”, “mango”, “melon”, “metallic”, “molasses”, “mushroom”, “musk”, “nutmeg”, “orange”, “orange flower”, “orris”, “passionfruit”, “patchouli”, “peach”, “pear”, “pepper”, “peppermint”, “pineapple”, “raspberry”, “resinous”, “rhubarb”, “rose”, “rosemary”, “sandalwood”, “spearmint”, “strawberry”, “tea”, “terpenic”, “thyme”, “tonka”, “vanilla”, “vetiver”, “violet”, “wintergreen”, “ylang ylang”, and “zest”.

This selection of flavour families and flavour descriptors allows the skilled flavourist to characterize the flavour of all flavour ingredients contained in the flavourist's palette. Nevertheless, for the trained flavourist, reading the contents of this specification as a whole together with their common general knowledge, it would not present undue burden to modify part or all of this vocabulary around which there is subjectivity, and such modification would not impact the selection of flavour ingredients useful to positively impact the perception of happiness.

Specific examples of fruity-strawberry ingredients, fruity-raspberry ingredients, fruity-apple ingredients, fruity-banana ingredients, floral-freesia ingredients, floral-lily of the valley ingredients, animalic-butyric ingredients, fruity-pineapple ingredients, fruity-peach ingredients, herbal-spearmint ingredients, citrus-orange ingredients, spicy-cinnamon ingredients, floral-jasmine ingredients, cooling ingredients, aromatic-wintergreen ingredients, citrus-lime ingredients, citrus-mandarin ingredients, citrus-grapefruit ingredients, spicy-pepper ingredients, spicy-clove ingredients, herbal-rosemary ingredients, green-grass ingredients, woody-resinous ingredients, herbal-coniferous ingredients, sweet-vanilla ingredients, sweet-coconut ingredients, sweet-cooked sugar ingredients, fruity-passionfruit ingredients, and herbal-thyme ingredients, respectively, will be provided below.

Throughout this application, the term “oil” is meant to encompass fully natural essential oils and extracts, as well as oils derived from natural essential oils and extracts, and modified essential oils and extracts that may comprise additional ingredients; irrespective of the extraction method.

The term “oil” is meant to further also encompass any reconstitution or mixture of ingredients that provides a similar odour impression to the corresponding essential oil.

The present invention further provides oral care flavour compositions for improving the happiness state of a human subject.

The oral care flavour composition comprises:
a) at least one HMP oral care flavour ingredient;
b) at least about 15% by weight in total of HMP and/or HMI and/or HMR oral care flavour ingredients;
c) optionally up to about 85% by weight in total of other oral care flavour ingredients, provided the following conditions are met:
   (c1) PEPPERMINTs+INVs<75%
   (c2) HMPs+HMIs>RELs
   (c3) HMPs+HMRs>INVs
   (c4) HMPs/(HMPs+RELs+INVs)≥0.3

In the above formulation guidelines, all percentages are based on total weight of the oral care flavour ingredients constituting the oral care flavour composition.

HMPs indicates the sum of percentages of HMP oral care flavour ingredients; HMIs indicates the sum of percentages of HMI oral care flavour ingredients; HMRs indicates the sum of percentages of HMR oral care flavour ingredients; PEPPERMINTs indicates the sum of percentages of PEPPERMINT oral care flavour ingredients; INVs indicates the sum of percentages of INV oral care flavour ingredients; and RELs indicates the sum of percentages of REL oral care flavour ingredients.

The symbol indicates at least equal to.

The present invention is based on extensive testing of oral care flavour ingredients, by consumer testing and measurement of brain activity using fNIRS. Statistical analysis of the resulting data has allowed classifying the oral care flavour materials into different categories:
HMP comprises ingredients or bases strongly associated with happy moods;
HMI comprises ingredients that may support both happy and invigorating moods;
HMR comprises ingredients that may support both happy and relaxing moods;
PEPPERMINT oral care flavour ingredients have a peppermint organoleptic attribute;
INV comprises ingredients that strongly support invigorating moods; and
REL comprises ingredients that strongly support relaxing moods.

It must be emphasized that these designations are relevant to ingredients as used by one skilled in the art (e.g. a flavourist) under the dosage and pattern constraints disclosed here.

The HMP oral care flavour ingredients are selected from the group consisting of fruity-strawberry ingredients, fruity-raspberry ingredients, fruity-apple ingredients, fruity-banana ingredients, floral-freesia ingredients, floral-lily of the valley ingredients, animalic-butyric ingredients, acetic acid, and mixtures thereof.

Fruity-strawberry ingredients include, but are not limited to, e.g. benzyl 3-phenylprop-2-enoate (benzyl cinnamate), ethyl butanoate (ethyl butyrate), ethyl 2-methylpropionate (ethyl isobutyrate), ethyl 3-phenylprop-2-enoate (ethyl cinnamate), methyl 3-phenylprop-2-enoate (methyl cinnamate), ethyl 3-phenyloxirane-2-carboxylate (ethyl phenyl glycidate), ethyl 3-methyl-3-phenyloxirane-2-carboxylate (ethyl methyl phenyl glycidate), 2-phenylethyl butanoate (phenyl ethyl butyrate), benzyl butanoate (benzyl butyrate), and ethyl 3-methylbutanoate (ethyl isovalerate).

Fruity-raspberry ingredients include, but are not limited to, e.g. 4-(4-hydroxyphenyl)butan-2-one (raspberry ketone), and methyoxy phenyl butanone.

Fruity-apple ingredients include, but are not limited to, e.g. ethyl 2-methylbutanoate (ethyl 2-methyl butyrate), ethyl 3-oxobutanoate (ethyl aceto acetate), ethyl 2-methylpentanoate (manzanate), (Z)-hex-3-enyl acetate (cis-3-hexenyl acetate), (E)-2-hexenal (trans-2-hexenal), diethyl propanedioate (diethyl malonate), and [(E)-hex-2-enyl] acetate (trans-2-hexenyl acetate).

Fruity-banana ingredients include, but are not limited to, e.g. pentyl propanoate (amyl propionate), 3-methylbutyl 3-methylbutanoate (iso amyl iso valerate), 6-methylhept-5-en-2-one (methyl heptenone), 3-methylbutyl butanoate (iso amyl butyrate), 3-methylbutyl acetate (iso amyl acetate), pentyl butanoate (amyl butyrate), butyl acetate, and 2-methylpropyl acetate (iso butyl acetate).

Floral-freesia ingredients include, but are not limited to, e.g. 3,7-dimethylocta-1,6-dien-3-ol (linalool), 2-(4-methyl-1-cyclohex-3-enyl)propan-2-yl acetate (terpinyl acetate), 2-(4-methyl-1-cyclohex-3-enyl)propan-2-ol (terpineol alpha), and 3,7-dimethylocta-1,6-dien-3-yl (E)-3-phenylprop-2-enoate (linalyl cinnamate).

Floral-lily of the valley ingredients include, but are not limited to, e.g. 7-hydroxy-3,7-dimethyloctanal (hydroxycitronellal), 2-methyl-3-(4-propan-2-ylphenyl)propanal (cyclamen aldehyde), 3-(1,3-benzodioxol-5-yl)-2-methylpropanal (tropional), and 2,6-dimethylhept-5-enal (melonal).

Animalic-butyric ingredients include, but are not limited to, e.g. butanoic acid (butyric acid), octanoic acid, 2-methylbutanoic acid (2 methyl butyric acid), and hexanoic acid (caproic acid).

The HMI oral care flavour ingredients are selected from the group consisting of citrus-orange ingredients, spicy-cinnamon ingredients, floral-jasmine ingredients, cooling ingredients, aromatic-wintergreen ingredients, lime terpenes, lime terpeneless, and mixtures thereof.

Citrus-orange ingredients include, but are not limited to, e.g. of orange oil and orange terpenes.

Spicy-cinnamon ingredients include, but are not limited to, e.g. cassia oils, 3-phenylprop-2-enal (cinnamic aldehyde), and 2-methyl-3-phenylprop-2-enal (methyl cinnamic aldehyde).

Floral-jasmine ingredients include, but are not limited to, e.g. Jasmin oil, methyl 3-oxo-2-pentylcyclopentaneacetate (methyl dihydrojasmonate), 3-methyl-2-[(Z)-pent-2-enyl]cyclopent-2-en-1-one (cis-jasmone), 2-(phenylmethylidene)octanal (hexyl cinnamic aldehyde), benzyl propanoate (benzyl propionate), [(Z)-hex-3-enyl] 2-hydroxybenzoate (cis-3-hexenyl salicylate), benzyl 2-hydroxybenzoate (benzyl salicylate), 3,7,11-trimethyldodeca-2,6,10-trien-1-ol (farnesol), 3,7,11-trimethyldodeca-1,6,10-trien-3-ol (nerolidol), and 2-benzylideneheptanal (amyl cinnamic aldehyde).

Cooling ingredients include, but are not limited to, e.g. N-(4-cyanomethylphenyl) p-menthane carboxamide (Evercool 180), 2-isopropyl-5-methyl-N-(2-(pyridin-2-yl)ethyl)cyclohexane carboxamide (Evercool 190), 3-(5-methyl-2-propan-2-ylcyclohexyl)oxypropane-1,2-diol (Coolact P), (1R,2R,5S)-5-methyl-2-prop-1-en-2-ylcyclohexan-1-ol (Coolact 10), ethyl 2-[[(1R,2S,5R)-5-methyl-2-propan-2-ylcyclohexanecarbonyl]amino]acetate (WS-5), (1R,2S,5R)—N-(4-methoxy-phenyl)-5-methyl-2-(1-methylethyl)cyclohexanecarboxamide (WS-12), N-ethyl-5-methyl-2-propan-2-ylcyclohexane-1-carboxamide (WS-3), N,2,3-trimethyl-2-propan-2-ylbutanamide (WS-23), (E)-3-benzo(d)[1,3]dioxol-5-yl)-N,N-diphenylacrylamide (Icool MC6), 2-(p-tolyloxy)-N-(1H-pyrazol-5-yl)-N-((thiophen-2-yl)methyl)acetamide (Freezestorm), [(1R,2S,5R)-5-methyl-2-propan-2-ylcyclohexyl] 2-hydroxypropanoate (Frescolat ML), 2-isopropyl-5-methylcyclohexyloxy-carbonyloxy-2-hydroxypropane (Frescolat MGC), 2-hydroxypropyl (5-methyl-2-propan-2-ylcyclohexyl) carbonate (Frescolat MPC), 9-Methyl-6-(1-methylethyl)-1,4-dioxaspiro-[4,5]decan-2-methanol (Frescolat MGA), menthyl lactate, mono-menthyl succinate, menthyl methyl ether, (2E)-3-(1,3-benzodioxol-5-yl)-N,N-diphenylacrylamide (iCool MC 6), 1-methyl-3-hydroxybutyrate, 2-{[5-methyl-2-(propan-2-yl)cyclohexyl]oxy}ethanol (Coolact 5), and isomeric mix of 2-(2-hydroxypropan-2-yl)-5-methylcyclohexan-1-ol (Coolact 38D).

Aromatic-wintergreen ingredients include, but are not limited to, e.g. ethyl 2-hydroxybenzoate (ethyl salicylate), methyl 2-hydroxybenzoate (methyl salicylate), and wintergreen oil.

The HMR oral care flavour ingredients are selected from the group consisting of fruity-pineapple ingredients, fruity-peach ingredients, herbal-spearmint ingredients, lemon oil, lemon oil reconstitutions, and mixtures thereof.

Fruity-pineapple ingredients include, but are not limited to, e.g. pentyl hexanoate (amyl caproate), prop-2-enyl 3-cyclohexylpropanoate (allyl cyclohexyl propionate), 3-methylbutyl hexanoate (isoamyl caproate), methyl hexanoate, ethyl hexanoate, ethyl heptanoate, 2-phenylethyl 2-methylpropanoate (phenyl ethyl isobutyrate), and prop-2-enyl propanoate (allyl propionate).

Fruity-peach ingredients include, but are not limited to, e.g. 5-heptyldihydrofuran-2(3H)-one (undecalactone gamma), 5-hexyloxolan-2-one (decalactone gamma), 5-octyloxolan-2-one (dodecalatone gamma), and 6-heptyloxan-2-one (dodecalatone delta).

Herbal-spearmint ingredients include, but are not limited to, e.g. spearmint oils, (1R,5S)-2-methyl-5-prop-1-en-2-ylcyclohex-2-en-1-ol and (1R,5R)-2-methyl-5-prop-1-en-2-ylcyclohex-2-en-1-ol (trans- and cis-carveol) and (5R)-2-methyl-5-prop-1-en-2-ylcyclohex-2-en-1-one (L-Carvone).

The PEPPERMINT oral care flavour ingredients include, but are not limited to, e.g. peppermint oil, L- and D/L-2-isopropyl-5-methylcyclohexanol (L- and D/L-menthol), [(1R,2S,5R)-5-methyl-2-propan-2-ylcyclohexyl] acetate (menthyl acetate), 2-butan-2-ylcyclohexan-1-one (freskomenthe), 5-methyl-2-prop-1-en-2-ylcyclohexan-1-ol (isopulegol), D/L- and L-2-isopropyl-5-methylcyclohexanone (D/L- and L-menthone), L- and D/L-2-isopropyl-5-methylcyclohexanone (L- and racemic isomenthone), and mixtures thereof.

The INV oral care flavour ingredients are selected from the group consisting of citrus-lime ingredients (excluding lime terpenes and lime terpeneless), citrus-mandarin ingredients, citrus-grapefruit ingredients, spicy-pepper ingredients, spicy-clove ingredients, herbal-rosemary ingredients, green-grass ingredients, woody-resinous ingredients, herbal-coniferous ingredients, bergamot oils, 3,7-dimethylocta-1,6-dien-3-yl acetate (linalyl acetate), 3,7-dimethylocta-1,6-dien-3-yl 2-methylpropanoate (linalyl iso butyrate), (E)-3,7-dimethylocta-2,6-dienal (citral), lemongrass, Litsea cubeba oils, and mixtures thereof.

Citrus-lime ingredients (excluding lime terpenes and lime terpeneless) include, but are not limited to, e.g. lime oil, 1-methyl-4-propan-2-ylidenecyclohexene (terpinolene), (E)-3,7-dimethylocta-1,3,6-triene (ocimene), and 1-methyl-4-propan-2-ylcyclohexa-1,4-diene (terpinene gamma).

Citrus-mandarin ingredients include, but are not limited to, e.g. mandarin oil, tangerine oil, and methyl 2-methylaminobenzoate (dimethyl anthranilate).

Citrus-grapefruit ingredients include, but are not limited to, e.g. grapefruit oil.

Spicy-pepper ingredients include, but are not limited to, e.g. ginger oil, nutmeg oil, olibanum oil, cardamom oil, pepper oil, and (4Z)-4,11,11-trimethyl-8-methylenebicyclo[7.2.0]undec-4-ene (caryophyllene).

Spicy-clove ingredients include, but are not limited to, e.g. clove oils, 4-allyl-2-methoxyphenol (eugenol), and (E)-2-methoxy-4-(prop-1-en-1-yl)phenol (isoeugenol).

Herbal-rosemary ingredients include, but are not limited to, e.g. rosemary oil, 1,7,7-trimethylbicyclo[2.2.1]heptan-2-one (camphor), and 2-(5-methyl-5-vinyltetrahydro-2-furanyl)-2-propanol (linalool oxide).

Green-grass ingredients include, but are not limited to, e.g. (Z)-hex-3-en-1-ol (cis-3-hexenol), [(Z)-hex-3-enyl] 2-methylpropanoate (cis-3-isobutyrate), and (E)-hex-2-en-1-ol (trans-2-hexenol).

Woody-resinous ingredients include, but are not limited to, e.g. pine oil and pine oil reconstitutions.

Herbal-coniferous ingredients include, but are not limited to, e.g. juniper berry oil, 7-methyl-3-methylideneocta-1,6-diene (myrcene), 4,7,7-trimethylbicyclo[3.1.1]hept-3-ene (pinene alpha), 7,7-dimethyl-4-methylidenebicyclo[3.1.1]heptane (pinene beta), [(1R,4S,6R)-1,7,7-trimethyl-6-bicyclo[2.2.1]heptanyl] acetate (iso bornyl acetate), and 1,3,3-trimethylbicyclo[2.2.1]heptan-2-ol (fenchyl alcohol).

The REL oral care flavour ingredients are selected from the group consisting of sweet-vanilla ingredients, sweet-coconut ingredients, sweet-cooked sugar ingredients, fruity-passionfruit ingredients, herbal-thyme ingredients, sage oils and reconstitutions, clary sage oils and reconstitutions, camomile oils and reconstitutions, lavender oils and reconstitutions, and mixtures thereof.

Sweet-vanilla ingredients include, but are not limited to, e.g. 4-hydroxy-3-methoxybenzaldehyde (vanillin), 3-ethoxy-4-hydroxybenzaldehyde (vanilla or ethyl vanillin), (4-formyl-2-methoxyphenyl)-2-methylpropanoate (isobutavan), and 2-methoxy-4-methylphenol (cresol).

Sweet-coconut ingredients include, but are not limited to, e.g. 6-pentyloxan-2-one (decalactone delta), 5-pentyloxolan-2-one (nonalactone gamma), 5-propyloxolan-2-one (heptalactone gamma), 5-ethyloxolan-2-one (hexalactone gamma), and 5-butyloxolan-2-one (octalactone gamma).

Sweet-cooked sugar ingredients include, but are not limited to, e.g. 2-ethyl-4-hydroxy-5-methyl-furan-3-one (homofuranol), 3-hydroxy-2-methylpyran-4-one (maltol), 2-ethyl-3-hydroxypyran-4-one (ethyl maltol), and 4-hydroxy-2,5-dimethylfuran-3-one (furaneol).

Fruity-passionfruit ingredients include, but are not limited to, e.g. (2R,4S)-2-methyl-4-propyl-1,3-oxathiane (oxane).

Herbal-thyme ingredients include, but are not limited to, e.g. thyme oils and reconstitutions, 5-methyl-2-propan-2-ylphenol (thymol), and origanum oils and reconstitutions.

Oral care flavour compositions of the present invention may further contain substantially flavourless and odourless ingredients. In the context of the present invention, “substantially flavourless and odourless means that the ingredient has no odour or aroma or taste, or that its odour, aroma or taste is weak and often barely perceptible. These substantially odourless and tasteless ingredients include excipients conventionally used in conjunction with flavour ingredients in flavour compositions, for example carrier materials, and other auxiliary agents commonly used in the art, e.g. solvents, such as isopropyl myristate (IPM), benzyl benzoate (BB), propylene glycol (PG) and triethyl citrate (TEC), Iso Propyl alcohol (IPA) Triacetin (TRI), Tween 20 (TWE); mineral oils and vegetable oils; and other oils such as castor seed oil.

The oral care flavour compositions of the present invention may be presented in the form of free-oil, or they may be encapsulated. Several encapsulating media are known in the art for encapsulating oral care flavour compositions. Particular encapsulating media include microcapsules formed of natural or modified natural polymers, such as polysaccharides or proteins.

The above definition of the oral care flavour compositions of the present invention provides sufficient freedom in formulation to permit consideration of the hedonic properties of the composition. The invention can thus enable formulation of oral care flavour compositions that evoke happiness and also have good hedonic properties.

The present invention describes how to formulate reliably oral care flavour compositions which are likely to induce or be associated with positive, happy moods and emotions. The effects are sufficiently pronounced that they can be measured reliably and reproducibly. The oral care flavour compositions made according to the teachings disclosed herein can be hedonically pleasant, suitable for a wide range of consumer products, and of sufficient pleasantness/acceptability that they would be appropriate even if they did not possess added functionality.

Oral care flavour compositions in accordance with the invention have been found:
1. to promote positive mood states, such as happy, uplifted, enthusiastic, ecstatic, light-hearted, carefree, euphoric, excited, delighted, playful, optimistic and fulfilled: test subjects have reported that they feel happier after orally applying or using consumer products incorporating the oral care flavour compositions, and that the products themselves taste happier;
2. not to promote negative mood states, such as depressing, stressful, irritating, or bored mood states.

Increasing the level of “happy” ingredients, such as HMP, HMI, and HMR, increases the likelihood that the oral care flavour would have a suitable character to enhance the state of happiness. Other ingredients reduce the likelihood that the benefit will be achieved as their level in the oral care flavour composition is increased, in particular ingredients that highly contribute to invigorating (INV) or relaxing (REL) moods.

In an embodiment, the oral care flavour composition comprises at least about 17%, more preferably at least about 20%, by weight in total of HMP and/or HMI and/or HMR oral care flavour ingredients.

In an embodiment, the oral care flavour composition comprises at least one HMP and/or HMI and/or HMR oral care flavour ingredient selected from one or more of the following groups:
one or more fruity-strawberry ingredients selected from the group consisting of benzyl 3-phenylprop-2-enoate (benzyl cinnamate), ethyl butanoate (ethyl butyrate), ethyl 2-methylpropionate (ethyl isobutyrate), ethyl 3-phenylprop-2-enoate (ethyl cinnamate), methyl 3-phenylprop-2-enoate (methyl cinnamate), ethyl 3-phenyloxirane-2-carboxylate (ethyl phenyl glycidate), ethyl 3-methyl-3-phenyloxirane-2-carboxylate (ethyl methyl phenyl glycidate), 2-phenylethyl butanoate (phenyl ethyl butyrate), benzyl butanoate (benzyl butyrate), ethyl 3-methylbutanoate (ethyl isovalerate), and mixtures thereof;
one or more fruity-raspberry ingredients selected from the group consisting of 4-(4-hydroxyphenyl)butan-2-one (raspberry ketone), methyoxy phenyl butanone, and mixtures thereof;
one or more fruity-apple ingredients selected from the group consisting of ethyl 2-methylbutanoate (ethyl 2-methyl butyrate), ethyl 3-oxobutanoate (ethyl aceto acetate), ethyl 2-methylpentanoate (manzanate), (Z)-hex-3-enyl acetate (cis-3-hexenyl acetate), (E)-2-hexenal (trans-2-hexenal), diethyl propanedioate (diethyl malonate), [(E)-hex-2-enyl]acetate (trans-2-hexenyl acetate), and mixtures thereof;
one or more fruity-banana ingredients selected from the group consisting of pentyl propanoate (amyl propionate), 3-methylbutyl 3-methylbutanoate (iso amyl iso valerate), 6-methylhept-5-en-2-one (methyl heptenone), 3-methylbutyl butanoate (iso amyl butyrate), 3-methylbutyl acetate (iso amyl acetate), pentyl butanoate (amyl butyrate), butyl acetate, 2-methylpropyl acetate (iso butyl acetate), and mixtures thereof;
one or more floral-freesia ingredients selected from the group consisting of 3,7-dimethylocta-1,6-dien-3-ol (linalool), 2-(4-methyl-1-cyclohex-3-enyl)propan-2-yl acetate (terpinyl acetate), 2-(4-methyl-1-cyclohex-3-enyl)propan-2-ol (terpineol alpha), 3,7-dimethylocta-1,6-dien-3-yl (E)-3-phenylprop-2-enoate (linalyl cinnamate), and mixtures thereof;
one or more floral-lily of the valley ingredients selected from the group consisting of 7-hydroxy-3,7-dimethyloctanal (hydroxycitronellal), 2-methyl-3-(4-propan-2-ylphenyl)propanal (cyclamen aldehyde), 3-(1,3-benzodioxol-5-yl)-2-methylpropanal (tropional), 2,6-dimethyl-hept-5-enal (melonal), and mixtures thereof;
one or more animalic-butyric ingredients selected from the group consisting of butanoic acid (butyric acid), octanoic acid, 2-methylbutanoic acid (2 methyl butyric acid), hexanoic acid (caproic acid), and mixtures thereof.
one or more citrus-orange ingredients selected from the group consisting of orange oil, orange terpenes, and mixtures thereof;
one or more spicy-cinnamon ingredients selected from the group consisting of cassia oils, 3-phenylprop-2-enal (cinnamic aldehyde), 2-methyl-3-phenylprop-2-enal (methyl cinnamic aldehyde), and mixtures thereof;
one or more cooling ingredients selected from the group consisting of N-(4-cyanomethylphenyl) p-menthane carboxamide (Evercool 180), 2-isopropyl-5-methyl-N-(2-(pyridin-2-yl)ethyl)cyclohexane carboxamide (Evercool 190), 3-(5-methyl-2-propan-2-yl-cyclohexyl)oxypropane-1,2-diol (Coolact P), (1R,2R,5S)-5-methyl-2-prop-1-en-2-ylcyclo-hexan-1-ol (Coolact 10), ethyl 2-[[(1R,2S,5R)-5-methyl-2-propan-2-ylcyclohexane-carbonyl]amino]acetate (WS-5), (1R,2S,5R)—N-(4-methoxyphenyl)-5-methyl-2-(1-methyl-ethyl)cyclohexanecarboxamide (WS-12), N-ethyl-5-methyl-2-propan-2-ylcyclohexane-1-carboxamide (WS-3), N,2,3-trimethyl-2-propan-2-ylbutanamide (WS-23), (E)-3-benzo(d) [1,3]dioxol-5-yl)-N,N-diphenylacrylamide (Icool MC6), 2-(p-tolyloxy)-N-(1H-pyrazol-5-yl)-N-((thiophen-2-yl)methyl)acetamide (Freezestorm), [(1R,2S,5R)-5-methyl-2-propan-2-ylcyclohexyl] 2-hydroxypropanoate (Frescolat ML), 2-isopropyl-5-methylcyclohexyloxycarbonyl-oxy-2-hydroxypropane (Frescolat MGC), 2-hydroxypropyl (5-methyl-2-propan-2-ylcyclohexyl) carbonate (Frescolat MPC), 9-Methyl-6-(1-methylethyl)-1,4-dioxaspiro-[4,5]decan-2-methanol (Frescolat MGA), menthyl lactate, mono-menthyl succinate, menthyl methyl ether, (2E)-3-(1,3-benzodioxol-5-yl)-N,N-diphenylacrylamide (iCool MC 6), 1-methyl-3-hydroxybutyrate, 2-{[5-methyl-2-(propan-2-yl)cyclohexyl]oxy}ethanol (Coolact 5), isomeric mix of 2-(2-hydroxypropan-2-yl)-5-methylcyclohexan-1-ol (Coolact 38D), and mixtures thereof;
one or more aromatic-wintergreen ingredients selected from the group consisting of ethyl 2-hydroxybenzoate (ethyl salicylate), methyl 2-hydroxybenzoate (methyl salicylate), wintergreen oil, and mixtures thereof;
one or more fruity-pineapple ingredients selected from the group consisting of pentyl hexanoate (amyl caproate), prop-2-enyl 3-cyclohexylpropanoate (allyl cyclohexyl propionate), 3-methylbutyl hexanoate (isoamyl caproate), methyl hexanoate, ethyl hexanoate, ethyl heptanoate, 2-phenylethyl 2-methylpropanoate (phenyl ethyl isobutyrate), prop-2-enyl propanoate (allyl propionate), and mixtures thereof;
one or more fruity-peach ingredients selected from the group consisting of 5-heptyldihydrofuran-2(3H)-one (undecalactone gamma), 5-hexyloxolan-2-one (decalactone gamma), 5-octyloxolan-2-one (dodecalatone gamma), 6-heptyloxan-2-one (dodecalatone delta), and mixtures thereof; and
one or more herbal-spearmint ingredients selected from the group consisting of spearmint oils, (1R,5S)-2-methyl-5-prop-1-en-2-ylcyclohex-2-en-1-ol and (1R,5R)-2-methyl-5-prop-1-en-2-ylcyclohex-2-en-1-ol (trans- and cis-carveol) and (5R)-2-methyl-5-prop-1-en-2-ylcyclohex-2-en-1-one (L-Carvone), and mixtures thereof.

In an embodiment, the amounts of the ingredients are chosen such that PEPPERMINTs+INVs<70%, more preferably PEPPERMINTs+INVs<65%, and most preferably PEPPERMINTs+INVs<60%.

In an embodiment, the amounts of the ingredients are chosen such that HMPs/(HMPs+RELs+INVs)≥0.35, more preferably HMPs/(HMPs+RELs+INVs)≥0.40, and most preferably HMPs/(HMPs+RELs+INVs)≥0.45.

Another aspect of the invention relates to a method of delivering positive mood benefits, particularly happiness, to human subjects, comprising delivering the oral care flavour composition to said human subjects. For instance, the oral care flavour may be delivered in a consumer product.

Therefore, the present invention also provides a consumer product comprising the oral care flavour composition of the invention.

The oral care flavour compositions of the present invention may be used to impart desirable aroma/flavour impressions on all manner of consumer products, such as for instance toothpastes, mouthwashes, chewing gums, lozenges, breath freshening powders/tablets, breath sprays, breath films, dental floss, dentrifices, denture care products, gels, mousse, creams, also over the counter or prescription products for the treatment of gingivitis, plaque, tartar, caries, infections, inflammations, swelling, bleeding and oral malodour.

Oral care consumer products comprise formulated mixtures of various functional ingredients depending on product format and use, such as, but not limited to, surfactants, emulsifiers, binders, fillers, abrasives, humectants, foaming agents, flavouring agents, sweetening agents, thickening agents colouring agents, alkali metal bicarbonate salts, fluoride ion sources, gums, gels, whitening/bleaching agents, titanium dioxide, stain removing agents, water, alcohol, enzymes, antibacterial agents, and solvents. These formulated mixtures are usually referred to as “bases”.

The proportion of flavour composition contained in such products may lie in a range from 0.01% (as for example in a mouthwash for children) to 10% (as for example in a floss) based on the total weight of the consumer product. The means of incorporating an oral care flavour composition into a consumer product is known. Existing techniques may be used for incorporating the oral care flavour composition directly into a product, or the oral care flavour composition may be absorbed on a carrier material and then admixed to the product.

Also included within the scope of the invention is a method of delivering positive mood benefits or happiness benefits to a subject, particularly a human, comprising administering to the subject an effective amount of an oral care flavour composition in accordance with the invention. The composition should be administered in an appropriate amount to produce a benefit (i.e. a suprathreshold amount) without causing irritation (i.e. a non-irritant amount). An appropriate effective amount of any given composition can be readily determined, e.g. by experiment. To be effective, the compositions should be administered for oral application by the subject.

Therefore, the present invention also provides a method of improving the happiness state of a human subject, comprising the step of providing an effective amount of the oral care flavour composition of the invention to the human subject.

In the context of the studies resulting in the present invention, several oral care flavour ingredients have been identified that are able to improve the happiness state of a human subject.

Therefore, the present invention also relates to the use of an oral care flavour ingredient for improving the happiness state of a human subject, wherein the oral care flavour ingredient is selected from the group consisting of:
one or more fruity-strawberry ingredients selected from the group consisting of benzyl 3-phenylprop-2-enoate (benzyl cinnamate), ethyl butanoate (ethyl butyrate), ethyl 2-methylpropionate (ethyl isobutyrate), ethyl 3-phenylprop-2-enoate (ethyl cinnamate), methyl 3-phenylprop-2-enoate (methyl cinnamate), ethyl 3-phenyloxirane-2-carboxylate (ethyl phenyl glycidate), ethyl 3-methyl-3-phenyloxirane-2-carboxylate (ethyl methyl phenyl glycidate), 2-phenylethyl butanoate (phenyl ethyl butyrate), benzyl butanoate (benzyl butyrate), ethyl 3-methylbutanoate (ethyl isovalerate), and mixtures thereof;
one or more fruity-raspberry ingredients selected from the group consisting of 4-(4-hydroxyphenyl)butan-2-one (raspberry ketone), methyoxy phenyl butanone, and mixtures thereof;
one or more fruity-apple ingredients selected from the group consisting of ethyl 2-methylbutanoate (ethyl 2-methyl butyrate), ethyl 3-oxobutanoate (ethyl aceto acetate), ethyl 2-methylpentanoate (manzanate), (Z)-hex-3-enyl acetate (cis-3-hexenyl acetate), (E)-2-hexenal (trans-2-hexenal), diethyl propanedioate (diethyl malonate), [(E)-hex-2-enyl]acetate (trans-2-hexenyl acetate), and mixtures thereof;
one or more fruity-banana ingredients selected from the group consisting of pentyl propanoate (amyl propionate), 3-methylbutyl 3-methylbutanoate (iso amyl iso valerate), 6-methylhept-5-en-2-one (methyl heptenone), 3-methylbutyl butanoate (iso amyl butyrate), 3-methylbutyl acetate (iso amyl acetate), pentyl butanoate (amyl butyrate), butyl acetate, 2-methylpropyl acetate (iso butyl acetate), and mixtures thereof;
one or more floral-freesia ingredients selected from the group consisting of 3,7-dimethylocta-1,6-dien-3-ol (linalool), 2-(4-methyl-1-cyclohex-3-enyl)propan-2-yl acetate (terpinyl acetate), 2-(4-methyl-1-cyclohex-3-enyl)propan-2-ol (terpineol alpha), 3,7-dimethylocta-1,6-dien-3-yl (E)-3-phenylprop-2-enoate (linalyl cinnamate), and mixtures thereof;
one or more floral-lily of the valley ingredients selected from the group consisting of 7-hydroxy-3,7-dimethyloctanal (hydroxycitronellal), 2-methyl-3-(4-propan-2-ylphenyl)propanal (cyclamen aldehyde), 3-(1,3-benzodioxol-5-yl)-2-methylpropanal (tropional), 2,6-dimethyl-hept-5-enal (melonal), and mixtures thereof;
one or more animalic-butyric ingredients selected from the group consisting of butanoic acid (butyric acid), octanoic acid, 2-methylbutanoic acid (2 methyl butyric acid), hexanoic acid (caproic acid), and mixtures thereof.
one or more citrus-orange ingredients selected from the group consisting of orange oil, orange terpenes, and mixtures thereof;
one or more spicy-cinnamon ingredients selected from the group consisting of cassia oils, 3-phenylprop-2-enal (cinnamic aldehyde), 2-methyl-3-phenylprop-2-enal (methyl cinnamic aldehyde), and mixtures thereof;
one or more cooling ingredients selected from the group consisting of N-(4-cyanomethylphenyl) p-menthane carboxamide (Evercool 180), 2-isopropyl-5-methyl-N-(2-(pyridin-2-yl)ethyl)cyclohexane carboxamide (Evercool 190), 3-(5-methyl-2-propan-2-yl-cyclohexyl)oxypropane-1,2-diol (Coolact P), (1R,2R,5S)-5-methyl-2-prop-1-en-2-ylcyclo-hexan-1-ol (Coolact 10), ethyl 2-[[(1R,2S,5R)-5-methyl-2-propan-2-ylcyclohexanecarbonyl]amino]acetate (WS-5), (1R,2S,5R)—N-(4-methoxyphenyl)-5-methyl-2-(1-methylethyl)cyclohexanecarboxamide (WS-12), N-ethyl-5-methyl-2-propan-2-ylcyclohexane-1-carboxamide (WS-3), N,2,3-trimethyl-2-propan-2-ylbutanamide (WS-23), (E)-3-benzo(d)[1,3]dioxol-5-yl)-N,N-diphenylacrylamide (Icool MC6), 2-(p-tolyloxy)-N-(1H-pyrazol-5-yl)-N-((thiophen-2-yl)methyl)acetamide (Freezestorm), [(1R,2S,5R)-5-methyl-2-propan-2-ylcyclohexyl] 2-hydroxypropanoate (Frescolat ML), 2-isopropyl-5-methylcyclohexyloxycarbonyloxy-2-hydroxypropane (Frescolat MGC), 2-hydroxypropyl (5-methyl-2-propan-2-ylcyclohexyl) carbonate (Frescolat MPC), 9-Methyl-6-(1-methylethyl)-1,4-dioxaspiro-[4,5]decan-2-methanol (Frescolat MGA), menthyl lactate, mono-menthyl succinate, menthyl methyl ether, (2E)-3-(1,3-benzodioxol-5-yl)-N,N-diphenylacrylamide (iCool MC 6), 1-methyl-3-hydroxybutyrate, 2-{[5-methyl-2-(propan-2-yl)cyclohexyl]oxy}ethanol (Coolact 5), isomeric mix of 2-(2-hydroxypropan-2-yl)-5-methylcyclohexan-1-ol (Coolact 38D), and mixtures thereof;
one or more aromatic-wintergreen ingredients selected from the group consisting of ethyl 2-hydroxybenzoate (ethyl salicylate), methyl 2-hydroxybenzoate (methyl salicylate), wintergreen oil, and mixtures thereof;
one or more fruity-pineapple ingredients selected from the group consisting of pentyl hexanoate (amyl caproate), prop-2-enyl 3-cyclohexylpropanoate (allyl cyclohexyl propionate), 3-methylbutyl hexanoate (isoamyl caproate), methyl hexanoate, ethyl hexanoate, ethyl heptanoate, 2-phenylethyl 2-methylpropanoate (phenyl ethyl isobutyrate), prop-2-enyl propanoate (allyl propionate), and mixtures thereof;
one or more fruity-peach ingredients selected from the group consisting of 5-heptyldihydrofuran-2(3H)-one (undecalactone gamma), 5-hexyloxolan-2-one (decalactone gamma), 5-octyloxolan-2-one (dodecalatone gamma), 6-heptyloxan-2-one (dodecalatone delta), and mixtures thereof; and
one or more herbal-spearmint ingredients selected from the group consisting of spearmint oils, (1R,5S)-2-methyl-5-prop-1-en-2-ylcyclohex-2-en-1-ol and (1R,5R)-2-methyl-5-prop-1-en-2-ylcyclohex-2-en-1-ol (trans- and cis-carveol) and (5R)-2-methyl-5-prop-1-en-2-ylcyclohex-2-en-1-one (L-Carvone), and mixtures thereof.

The present invention is further illustrated by means of the following non-limiting examples:

### EXAMPLE 1: FUNCTIONAL NEAR INFRARED SPECTROSCOPY TESTING

### Task

Six experiments, each one testing four oral care flavours, were run, analysing the flavours' potential to evoke specific moods. At the beginning of each session, participants were seated and the fNIRS cap was fitted. The experimenter checked for optimal contact between the optodes of the fNIRS system and the head of the participant. Upon setup of the fNIRS system and the recording of the initial baseline, the actual test began, where each participant tasted four different flavours in the form of a mouthwash, according to the timeline described below.

### Timeline

Participants completed four blocks, each block comprising either a relaxing, an invigorating, or a happy oral care flavour in the form of a mouthwash.

Each block began with the participant eating a cracker, followed by a 30 second rest, after which the participant had a drink of water to clear out any excess cracker in their mouth, which they could spit or swallow after rinsing the mouth, followed by another 30 second rest. Participants were then asked to use the mouthwash like they would use it at home, swirling it around their mouth and spitting it out into a bowl, followed by another 30 second rest. fNIRS testing was conducted during the 30 second rest.

A five-minute break was allowed between each block to ensure that there were no traces of the previous mouthwash left in the mouth before starting another trial and that the participants were able to taste all flavours properly. This five-minute break was extended if the participant asked for more time, or if fNIRS signals were not at the baseline level (e.g. due to heavy movement).

### Participants

Overall, for the six studies, 90 participants were tested. No specific participant exclusion criteria were implemented in the studies apart from being healthy.

### Neuroimaging Apparatus Details and Data Handling

Data pre-processing was performed using the NIRx NIRSLab software.

Recorded raw data was loaded in the NIRx NIRSLab software. After placing the event triggers on the continuous recorder data, time points were identified for each rest period in all four blocks, after which an automatic removal of discontinuities in the signal was performed.

The time series was truncated by cutting 12 segments in the continuous recording, each segments lasting 45 seconds (30 second post stimulus rest, plus 5 seconds before and 10 seconds after). Two 60 second eyes closed rest intervals were kept as meaningful data in order to analyse the shape of the haemodynamic response function (HRF). This curve models the increases and decreases in Hb concentration in a particular region following the presentation of a stimulus and can be exploited for fMRI-like analyses as the Statistical Parametric Mapping (SPM).

When signal was lost (detector saturation, meaning that too much light reaches the detector due to artefacts), the nearest-neighbour interpolation was applied. The longest interpolation interval was less than one second.

The visual inspection of the channels consisted in the removal of noisy data intervals exceeding arbitrary thresholds for the CV % (percentage of the coefficient of variance, representing the variance in the channel's data).

Data was filtered using a band-pass filter at the frequencies of 0.005-0.3 Hz.

The haemodynamic states were calculated by conversion of the light signal into Hb concentration. Pre-processed data (OxyHb, DeoxyHB, and Total Hb) were exported from the NirsLab interface and used for statistical analysis.

### Baseline Selection

Data for each participant was always normalized over a baseline interval to exalt differences due to the experimental paradigm rather than physiological (i.e. present independently from any experimental protocol, naturally existing). Different choices are currently considered in the literature, among which the choice of the full experiment recording as the dataset on which calculating the baseline, or a sub fragment of it. Choosing this latter approach to minimize the chance of losing meaningful fluctuations in the signal, one of the three control trials was selected as the baseline. The choice was fully randomized, but selected in advance.

### Analyses Performed

In the current study, two-way t-tests were performed to compare the effects on brain activity of each flavour vs. the “rinse with water” baseline immediately preceding the application of each flavour. This allowed to highlight differences in brain activity due exclusively to the application of the mouthwash, therefore allowing to define brain signatures for each mood analysed. These tests were repeated in four different time intervals: for the whole duration of the trials, i.e. 30 seconds, for the initial five seconds, for the 5-10 seconds interval and for the first ten seconds of the trials.

These analyses were originally performed including all the participants and averaging all twenty channels recorded. Of the twenty channels available, nine covered each hemisphere, while two recorded the activity over the inter-hemispheric fissure. Since a solid amount of the current literature assigns a different role to each hemisphere (see, for example, Hellige, 1993), it was decided to repeat all the previously described analyses in each brain hemisphere alone.

Finally, all the analyses were also repeated for the three fNIRS output parameters (OxyHb, DeoxyHb, Total Hb).

All the tests illustrated in Example 4 below were significant at a significance level of p s 0.05. All the results presented were also significant after correction for multiple comparisons through Bonferroni correction.

### EXAMPLE 2: MOOD PORTRAITS® TESTING

### Task

Mood Portraits® is a self-report nonverbal method using pictures to measure consumers' moods and emotional responses to fragrances and flavours. This method allows participants to express what they feel in response to tasting an oral care product by selecting images that match their feelings rather than verbalising and rating their thoughts and emotions.

The experimental protocol was as follows: Participants applied a series of two flavoured mouthwash products and, after rinsing their mouth and spitting out the product, they selected a number of pictures chosen from a set of thirty pictures to describe the flavour. The thirty pictures, printed in colour on A4 laminated sheets, were arranged on a display board. The number of pictures chosen by each participant to describe the flavours was not pre-determined: Each participant could choose as many as they wanted to describe each flavour. The minimum number of pictures they had to select was one.

The order of presentation for the flavoured mouthwashes was fully randomised and the pictures were arranged on four different boards to create a randomisation of the layout.

### Timeline

All participants applied two mouthwashes during a single session, for a total of four sessions in four consecutive days. They were asked to use the mouthwash as they would normally do at home, without any specific indication on the rinsing time. This allowed the participants to provide truer responses without any time pressure associated and to experience the product in the most natural way.

Participants were allowed breaks at their leisure to prevent any fatigue or carry over effect, and moved to the second mouthwash only when they considered themselves ready.

### Participants

For each test involving two mouthwashes, eighty healthy adults were asked to participate in the study. Participants were screened for olfactive and taste impairment, respiratory conditions or other personal conditions that could alter their sense of smell or taste (e.g. pregnancy or consumption of tobacco-based products, like cigarettes). No other selection criteria (i.e. handedness, age, gender, etc.) have been applied in the choice of the participants, since no relevant exclusion criteria have been identified prior to testing.

### EXAMPLE 3: COMPOSITIONS TESTED

| | | A | B | C | D | E | F | G | H |
|---|---|---|---|---|---|---|---|---|---|
| Ingredient | Group | wt % | wt % | wt % | wt % | wt % | wt % | wt % | wt % |
| BENZYL ACETATE | HMI | | | | | | | 0.005 | |
| BENZYL SALICYLATE | HMI | | | | 1.600 | | | | |
| CASSIA OIL | HMI | | | | | | | | 0.358 |
| CINNAMIC ALDEHYDE | HMI | | | | | | | | 4.137 |
| CIS JASMONE | HMI | | | | | | | 0.010 | |
| EVERCOOL 180 | HMI | | | | | | | 0.100 | |
| EVERCOOL 190 | HMI | | | | | | | 0.050 | |
| N-ethyl-5-methyl-2-propan-2-ylcyclohexane-1- | HMI | | | | | | | 1.000 | |
| carboxamide | | | | | | | | | |
| LIME TERPENES | HMI | | 10.00 | | | | | 3.000 | |
| METHYL DIHYDRO JASMONATE | HMI | | | | 4.400 | | | 0.025 | |
| ORANGE OIL | HMI | | 4.000 | 2.296 | | | | 17.50 | |
| 2-METHYL BUTYRIC ACID | HMP | | 0.100 | | | | | | |
| ACETIC ACID GLACIAL | HMP | | | | | | | 0.700 | |
| AMYL BUTYRATE | HMP | | | 0.028 | | | | | |
| BENZYL BUTYRATE | HMP | | 0.100 | | | | | | |
| BUTYL ACETATE | HMP | | | | | | | 2.500 | |
| CIS-3-HEXENYL ACETATE | HMP | | | | 0.020 | | | 0.012 | |
| CYCLAMEN ALDEHYDE | HMP | | | | 0.020 | | | | |
| ETHYL 2-METHYL BUTYRATE | HMP | | 0.200 | | | | | 1.200 | 0.090 |
| ETHYL ACETO ACETATE | HMP | | | | | | | | 1.768 |
| ETHYL AMYL KETONE | HMP | | | 0.050 | | | | | |
| ETHYL BUTYRATE | HMP | | 0.750 | 0.533 | | | | 0.120 | 0.250 |
| ETHYL ISO BUTYRATE | HMP | | | | | | | 1.000 | |
| ETHYL ISO VALERATE | HMP | | | | | | | 0.080 | |
| ISO AMYL ACETATE | HMP | | | 0.350 | | | | | 0.925 |
| ISO AMYL BUTYRATE | HMP | | 0.060 | | | | | | |
| LINALOL | HMP | | | | 1.200 | | | 0.040 | |
| MANZANATE | HMP | | | | | | | 5.000 | |
| METHOXY PHENYL BUTANONE | HMP | | | | | | | 0.095 | |
| METHYL CINNAMATE | HMP | | | | | | | 0.030 | |
| RASPBERRY KETONE | HMP | | | 0.040 | | | | 0.100 | |
| TERPINEOL ALPHA | HMP | | | | | 0.100 | | 0.004 | |
| TRANS 2 HEXENAL | HMP | | | | | | | | 0.050 |
| TRANS 2 HEXENYL ACETATE | HMP | | | | | | | | 0.250 |
| ALLYL HEPTANOATE | HMR | | | | | | | | 0.425 |
| ALLYL HEXANOATE | HMR | | | 2.383 | | | | | |
| CARVONE LAEVO | HMR | 3.000 | | 3.500 | | 13.40 | 12.00 | 3.000 | 2.740 |
| DECALACTONE GAMMA | HMR | | 0.200 | | | | | 0.045 | |
| DODECALACTONE GAMMA | HMR | | | | | | | 0.030 | |
| ETHYL HEPTANOATE | HMR | | | 0.200 | | | | 1.500 | |
| ETHYL HEXANOATE | HMR | | 0.250 | 1.217 | | | | 0.100 | 0.025 |
| LEMON OIL | HMR | | | 1.400 | 0.300 | | | | |
| SPEARMINT OIL | HMR | | 16.50 | | | 6.000 | 28.00 | 0.500 | 5.670 |
| UNDECALACTONE GAMMA | HMR | | | | 0.024 | | | | 0.050 |
| UNDECALACTONE DELTA | HMR | | | | | | | 0.004 | |
| ALCOHOL C6 | INV | | | | | | | | 0.550 |
| CARDAMOM OIL | INV | | | | 0.008 | | | | |
| CARYOPHYLLENE | INV | | | | | 0.200 | | | |
| CIS-3-HEXENOL | INV | | 0.400 | | | | | 0.025 | 0.025 |
| CIS-3-HEXENYL BUTYRATE | INV | | | | 0.020 | | | | |
| CITRAL | INV | | | | | | | 0.060 | |
| CLOVE LEAF OIL | INV | | | | | | | | 0.206 |
| EUGENOL | INV | | | | | | | 0.020 | |
| GINGER OIL | INV | | | 0.040 | | | | | |
| JUNIPER BERRY OIL | INV | | | | | | | 0.500 | |
| LIME OIL | INV | | 1.300 | | | | | | |
| LINALYL ACETATE | INV | | | | 1.000 | | | | |
| LINALYL ISO BUTYRATE | INV | | | | 0.200 | | | | |
| MANDARIN OIL | INV | | 0.040 | | 0.024 | | | | |
| MARJORAM OIL | INV | | 0.100 | | | | | | |
| NUTMEG | INV | | | | 0.040 | | | | |
| PEPPER BLACK | INV | 0.200 | | | | | | | |
| PIMENTO BERRY OIL | INV | | | | | | | | 0.137 |
| PINE OIL | INV | | | | 0.600 | | | | |
| MENTHOL LAEVO | PEPPERMINT | 29.80 | 40.80 | 55.00 | 57.00 | | 32.00 | 44.00 | 41.55 |
| MENTHOL RACEMIC | PEPPERMINT | | 15.00 | | | 41.30 | | 3.000 | |
| MENTHONE RACEMIC | PEPPERMINT | | | | | 3.200 | | | |
| PEPPERMINT OIL | PEPPERMINT | 58.00 | | 12.68 | 7.000 | 13.80 | 16.00 | 1.500 | 26.45 |
| ETHYL MALTOL | REL | | | | 0.100 | | 0.100 | 0.150 | 0.275 |
| FURANEOL | REL | | 0.020 | | | | | 0.008 | |
| HOMOFURONOL | REL | | | | | | | | 0.019 |
| MALTOL | REL | | | 0.035 | | | | | |
| NONALACTONE GAMMA | REL | | | 5.400 | | | | | |
| OCTALACTONE GAMMA | REL | | | 1.200 | | | | | |
| VANILLAL | REL | | | 2.900 | 0.200 | | | | 0.468 |
| VANILLIN | REL | | | 1.510 | 0.400 | | 0.100 | 0.100 | 0.533 |
| ACETYL METHYL CARBINOL | | | | | | | | | 0.001 |
| ALDEHYDE C6 (HEXANAL) | | | | | | | | | 0.055 |
| ANETHOLE | | 1.000 | 8.800 | 8.700 | 6.000 | 21.00 | 11.00 | 1.500 | 8.913 |
| ANISIC ALDEHYDE | | | | | 0.030 | | | | |
| BASIL OIL | | | 0.200 | | | | | | |
| BENZALDEHYDE | | | | | | | | | 0.003 |
| BENZOIN | | | | | 0.200 | | | | |
| BENZYL ALCOHOL | | | | | 0.099 | | | | 0.143 |
| CASSYRANE | | | | | 0.040 | | | | |
| CITRONELLOL | | | | | | | | 0.008 | |
| CORYLONE DRIED | | | | | | | | | 1.103 |
| DAMASCENONE | | | | | | | | 0.020 | |
| DAMASCONE ALPHA | | | | | | | | 0.020 | |
| DAMASCONE BETA | | | | | 0.020 | | | | 0.010 |
| DAVANA | | | | | | | | 0.040 | |
| DIMETHYL BENZYL CARBINYL BUTYRATE | | | | | | | | 0.008 | |
| ETHYL ACETATE | | | | | | | | 0.250 | |
| ETHYL LEVULINATE | | | | | | | | | 0.069 |
| ETHYL NONANOATE | | | | 0.050 | | | | | |
| ETHYL OXYHYDRATE | | | 1.000 | | | | | | |
| ETHYL PROPIONATE | | | | | | | | 0.250 | |
| EUCALYPTOL | | 8.000 | | 0.400 | | | | 1.000 | |
| EUCALYPTUS GLOBULUS | | | | | | 1.000 | | | |
| HELIOTROPIN | | | | 0.080 | 0.160 | | | | |
| HEXYL ACETATE | | | | | | | | | 0.500 |
| INDOLE | | | | | 0.001 | | | | |
| IONONE BETA | | | | | 0.900 | | | | |
| ISO AMYL ALCOHOL | | | | | | | | 0.050 | |
| ISO PROPYL ALCOHOL | | | | | | | | 3.000 | |
| ISOPROPYL MYRISTATE | | | | | 10.00 | | | | |
| LACTIC ACID | | | | | | | | 0.020 | |
| METHYL ANTHRANILATE | | | | | 0.002 | | | | |
| PATCHOULI OIL | | | | | 0.240 | | | | |
| PROPYLENE GLYCOL | | | 0.180 | 0.010 | 8.120 | | 0.800 | 0.072 | 2.228 |
| RHODINOL | | | | | | | | 0.015 | |
| ROSE OXIDE DEXTRO | | | | | 0.012 | | | | |
| STYRALLYL ACETATE | | | | | 0.020 | | | | 0.025 |
| TRIACETIN | | | | | | | | 6.614 | |
| YLANG YLANG OIL | | | | | | | | 0.020 | |
| Sum | | 100 | 100 | 100 | 100 | 100 | 100 | 100 | 100 |

| | | | | | | | | |
|---|---|---|---|---|---|---|---|---|
| Total % PEPPERMINT ingredients | 87.80 | 55.80 | 67.68 | 64.00 | 58.30 | 48.00 | 48.50 | 68.00 |
| Total % INV ingredients | 0.200 | 1.840 | 0.040 | 1.892 | 0.200 | | 0.605 | 0.918 |
| Total % HMI ingredients | | 14.00 | 2.296 | 6.000 | | | 21.69 | 4.495 |
| Total % HMP ingredients | | 1.210 | 1.001 | 1.240 | 0.100 | | 10.88 | 3.333 |
| Total % HMR ingredients | 3.000 | 16.95 | 8.699 | 0.324 | 19.40 | 40.00 | 5.179 | 8.910 |
| Total % REL ingredients | | 0.020 | 11.045 | 0.700 | | 0.200 | 0.330 | 1.372 |
| HMPs + HMRs + HMIs | 3.000 | 32.16 | 11.995 | 7.564 | 19.50 | 40.00 | 37.75 | 16.738 |
| PEPPERMINTs + INVs | 88.00 | 57.64 | 67.72 | 65.892 | 58.50 | 48.00 | 49.105 | 68.918 |
| HMPs + HMIs > RELs | No | Yes | No | Yes | Yes | No | Yes | Yes |
| HMPs + HMRs > INVs | Yes | Yes | Yes | No | Yes | Yes | Yes | Yes |
| HMPs/(HMPs + RELs + INVs) | 0.000 | 0.394 | 0.083 | 0.324 | 0.333 | 0.000 | 0.921 | 0.593 |
| RESULT | FAIL | PASS | FAIL | FAIL | PASS | FAIL | PASS | PASS |

Compositions A through H were subjected to fNIRS and/or Mood Portraits® testing. Among these, Compositions A, C, D, and F were comparative examples; while Compositions B, E, G, and H were oral care flavour compositions according to the present invention.

Ingredients contained in these compositions are specified in the above table.

### EXAMPLE 4: RESULTS OF FUNCTIONAL NEAR INFRARED SPECTROSCOPY TESTING

fNIRS testing of oral care flavour compositions A through H described in Example 3 was conducted according to the method described in Example 1. Water was used as the benchmark.

As a first level, the following eight conditions A1 through A8 were investigated:
A1. Channel 14 shows a statistically significant difference of Total Hb 0-10 seconds after the oral application;
A2. Channel 15 shows a statistically significant difference of Total Hb 0-10 seconds after the oral application;
A3. Channel 18 shows a statistically significant difference of Total Hb 0-10 seconds after the oral application;
A4. Channel 19 shows a statistically significant difference of Total Hb 0-10 seconds after the oral application;
A5. Channel 1 shows a statistically significant difference of Total Hb 0-10 seconds after the oral application;
A6. Channel 3 shows a statistically significant difference of Total Hb 0-10 seconds after the oral application;
A7. Channel 4 shows a statistically significant difference of Total Hb 0-10 seconds after the oral application;
A8. Channel 5 shows a statistically significant difference of Total Hb 0-10 seconds after the oral application.

Based on extensive testing, it had been determined that at least one of the above eight conditions A1 through A8 is met in case an oral care flavour composition provides a happy effect.

The results of the first level fNIRS testing are shown in the following table:

| Condition | | A | B | C | D | E | F | G | H |
|---|---|---|---|---|---|---|---|---|---|
| A1 | Test Flavour | 0.040 | 0.185 | 0.375 | 0.215 | 0.081 | 0.287 | 0.305 | −0.212 |
| | Benchmark | 0.311 | −0.015 | −0.233 | 0.023 | −0.269 | −0.169 | −0.528 | 0.072 |
| | Condition met | No | No | No | No | No | No | No | No |
| A2 | Test Flavour | −0.033 | −0.008 | −0.168 | 0.296 | 0.425 | −0.235 | 0.161 | 0.243 |
| | Benchmark | 0.015 | 0.067 | −0.148 | −0.261 | −0.050 | −0.360 | −0.745 | −0.385 |
| | Condition met | No | No | No | No | No | No | Yes | No |
| A3 | Test Flavour | 0.120 | 0.112 | 0.384 | 0.005 | 0.158 | −0.010 | 0.268 | 0.134 |
| | Benchmark | 0.359 | −1.013 | −0.575 | −0.354 | −0.208 | 0.402 | −0.238 | −0.314 |
| | Condition met | No | Yes | Yes | No | No | No | No | No |
| A4 | Test Flavour | −0.169 | −0.723 | 0.012 | 0.197 | 0.432 | 0.151 | 0.416 | −0.057 |
| | Benchmark | 0.346 | 0.376 | −0.085 | −0.047 | 0.225 | −0.201 | −0.534 | 0.335 |
| | Condition met | No | No | No | No | No | No | Yes | No |
| A5 | Test Flavour | 0.002 | −0.038 | 0.029 | 0.241 | 0.539 | 0.259 | −0.074 | −0.156 |
| | Benchmark | −0.205 | −0.019 | −0.362 | −0.307 | −0.183 | −0.420 | −0.147 | −0.492 |
| | Condition met | No | No | No | No | Yes | No | No | No |
| A6 | Test Flavour | −0.248 | −0.139 | 0.048 | 0.432 | 0.435 | 0.381 | 0.036 | 0.430 |
| | Benchmark | 0.158 | −0.063 | −0.032 | 0.017 | −0.159 | −0.315 | −0.064 | 0.303 |
| | Condition met | No | No | No | No | No | Yes | No | No |
| A7 | Test Flavour | 0.190 | 0.049 | 0.128 | 0.457 | 0.246 | 0.363 | 0.194 | 0.725 |
| | Benchmark | −0.001 | 0.315 | 0.267 | 0.426 | 0.168 | 0.101 | 0.150 | 0.185 |
| | Condition met | No | No | No | No | No | No | No | Yes |
| A8 | Test Flavour | 0.426 | −0.071 | 0.014 | 0.036 | 0.138 | 0.275 | 0.365 | −0.085 |
| | Benchmark | 0.806 | −0.080 | −0.221 | −0.181 | 0.086 | −0.183 | −0.788 | −0.185 |
| | Condition met | No | No | No | No | No | No | Yes | No |

| | | | | | | | | |
|---|---|---|---|---|---|---|---|---|
| Number of conditions A1-A8 | 0 | 1 | 1 | 0 | 1 | 1 | 3 | 1 |
| met | | | | | | | | |
| Happy effect? | NO | YES | YES | NO | YES | YES | YES | YES |

For those compositions that fulfilled the first level fNIRS requirements (Compositions B, C, and E-H), a further investigation of specific fNIRS channels and time points was conducted. Specifically, it was tested if one or more of the following nine further conditions B1 through B9 was met:
B1. Channel 14 shows a statistically significant difference of Oxy Hb 0-10 seconds after the oral application;
B2. Channel 16 shows a statistically significant difference of Oxy Hb 0-10 seconds after the oral application;
B3. Channel 18 shows a statistically significant difference of Oxy Hb 0-10 seconds after the oral application;
B4. Channel 20 shows a statistically significant difference of Oxy Hb 0-10 seconds after the oral application;
B5. Channel 2 shows a statistically significant difference of Oxy Hb 0-10 seconds after the oral application;
B6. Channel 3 shows a statistically significant difference of Oxy Hb 0-10 seconds after the oral application;
B7. Channel 4 shows a statistically significant difference of Oxy Hb 0-10 seconds after the oral application;
B8. Channel 5 shows a statistically significant difference of Oxy Hb 0-10 seconds after the oral application;
B9. Channel 6 shows a statistically significant difference of Oxy Hb 0-10 seconds after the oral application.

The results of this second level fNIRS testing are shown in the following table:

| Condition | | B | C | E | F | G | H |
|---|---|---|---|---|---|---|---|
| B1 | Test Flavour | 0.087 | 0.138 | 0.393 | 0.359 | 0.139 | −0.177 |
| | Benchmark | −0.044 | −0.221 | −0.153 | −0.108 | −0.549 | 0.147 |
| | Condition met | No | No | No | No | No | No |
| B2 | Test Flavour | 0.078 | −0.058 | 0.258 | −0.267 | 0.321 | −0.234 |
| | Benchmark | −0.288 | −0.426 | −0.222 | 0.288 | −0.977 | −0.113 |
| | Condition met | No | No | Yes | No | Yes | No |
| B3 | Test Flavour | −0.012 | 0.189 | 0.255 | 0.153 | 0.322 | 0.274 |
| | Benchmark | −0.907 | −0.573 | −0.191 | 0.548 | −0.204 | 0.032 |
| | Condition met | Yes | No | No | No | No | No |
| B4 | Test Flavour | 0.228 | −0.254 | 0.122 | 0.021 | 0.065 | −0.037 |
| | Benchmark | 0.186 | −0.129 | −0.035 | −0.261 | −0.031 | 0.724 |
| | Condition met | No | No | Yes | No | No | No |
| B5 | Test Flavour | −0.149 | 0.106 | 0.548 | −0.261 | 0.031 | −0.107 |
| | Benchmark | −0.090 | −0.461 | −0.221 | −0.278 | 0.010 | −0.349 |
| | Condition met | No | Yes | No | No | No | No |
| B6 | Test Flavour | −0.110 | −0.013 | 0.424 | 0.244 | 0.163 | 0.383 |
| | Benchmark | −0.163 | −0.117 | −0.135 | −0.156 | −0.237 | 0.046 |
| | Condition met | No | No | No | No | No | No |
| B7 | Test Flavour | 0.077 | 0.076 | 0.330 | 0.338 | 0.259 | 0.696 |
| | Benchmark | 0.176 | 0.170 | 0.160 | 0.175 | 0.170 | 0.115 |
| | Condition met | No | No | No | No | No | Yes |
| B8 | Test Flavour | −0.095 | −0.073 | 0.488 | 0.271 | 0.460 | 0.009 |
| | Benchmark | −0.007 | −0.430 | 0.229 | −0.118 | −0.910 | −0.156 |
| | Condition met | No | No | No | No | Yes | No |
| B9 | Test Flavour | −0.380 | −0.047 | 0.602 | 0.150 | 0.130 | 0.049 |
| | Benchmark | −0.405 | −0.220 | −0.049 | −0.242 | 0.027 | −0.314 |
| | Condition met | No | No | No | No | No | No |

| | | | | | | |
|---|---|---|---|---|---|---|
| Number of conditions B1-B9 met | 1 | 1 | 2 | 0 | 2 | 1 |
| Happy Effect? | Yes | Yes | Yes | No | Yes | Yes |

It was found that oral care flavour compositions led to a more pronounced improvement of the happiness state if at least at least one out of the nine conditions B1 through B9 was met. This was the case for Compositions B, C, E, G, and H.

An even better happy effect was observed for those oral care flavour compositions that also met at least one out of the following eight conditions C1 through C8:
C1. Channel 16 shows a statistically significant difference of Total Hb 5-10 seconds after the oral application;
C2. Channel 19 shows a statistically significant difference of Total Hb 5-10 seconds after the oral application;
C3. Channel 20 shows a statistically significant difference of Total Hb 5-10 seconds after the oral application;
C4. Channel 1 shows a statistically significant difference of Total Hb 5-10 seconds after the oral application;
C5. Channel 4 shows a statistically significant difference of Total Hb 5-10 seconds after the oral application;
C6. Channel 6 shows a statistically significant difference of Total Hb 5-10 seconds after the oral application;
C7. Channel 9 shows a statistically significant difference of Total Hb 5-10 seconds after the oral application;
C8. Channel 12 shows a statistically significant difference of Total Hb 5-10 seconds after the oral application.

The results are shown in the following table:

| Condition | | B | C | E | G | H |
|---|---|---|---|---|---|---|
| C1 | Test Flavour | 0.333 | 0.173 | 0.108 | 0.367 | −0.126 |
| | Benchmark | −0.610 | −0.190 | −0.302 | −0.826 | −0.116 |
| | Condition met | No | No | Yes | Yes | No |
| C2 | Test Flavour | −0.788 | −0.100 | 0.422 | 0.388 | −0.115 |
| | Benchmark | 0.256 | 0.013 | 0.199 | −0.488 | 0.361 |
| | Condition met | No | No | No | No | No |
| C3 | Test Flavour | 0.180 | −0.138 | 0.127 | 0.084 | 0.054 |
| | Benchmark | 0.034 | −0.120 | −0.056 | 0.008 | 0.588 |
| | Condition met | No | No | No | No | No |
| C4 | Test Flavour | −0.098 | 0.072 | 0.598 | −0.073 | 0.105 |
| | Benchmark | 0.051 | −0.300 | 0.104 | −0.092 | −0.449 |
| | Condition met | No | No | Yes | No | No |
| C5 | Test Flavour | −0.045 | 0.033 | 0.194 | 0.223 | 0.799 |
| | Benchmark | 0.220 | 0.337 | 0.117 | 0.180 | 0.123 |
| | Condition met | No | No | No | No | Yes |
| C6 | Test Flavour | −0.201 | 0.050 | 0.550 | 0.177 | 0.160 |
| | Benchmark | −0.672 | −0.266 | 0.088 | 0.068 | −0.184 |
| | Condition met | No | No | No | Yes | No |
| C7 | Test Flavour | 0.020 | 0.336 | 0.226 | 0.253 | 0.350 |
| | Benchmark | −0.361 | −0.505 | 0.048 | −0.152 | −0.353 |
| | Condition met | No | No | No | No | No |
| C8 | Test Flavour | 0.508 | 0.199 | 0.336 | 0.303 | 0.296 |
| | Benchmark | −0.183 | −0.306 | 0.217 | 0.451 | −0.293 |
| | Condition met | Yes | No | No | No | No |

| | | | | | |
|---|---|---|---|---|---|
| Number of conditions C1-C8 met | 1 | 0 | 2 | 2 | 1 |

It has been found that the additional criteria lead to an improved accuracy for predicting the effect on happiness achieved by the oral care flavour compositions. This was the case for Compositions B, E, G, and H. Consequently, the rules for preparing the oral care flavour compositions of the invention were devised such that the respective oral care flavour compositions pass even the highest level of fNIRS testing.

Furthermore, fNIRS testing shows very specific brain signatures at both group level (i.e. full brain and/or hemispherical averages) and at single channel level, making the validation test so thorough that only oral care flavour compositions and oral care flavour ingredients truly providing a sense of happiness in the participant can pass it.

Thus, the compositions of the present invention were found to be happy on the sub-conscious level.

### EXAMPLE 5: RESULTS OF MOOD PORTRAITS® TESTING

In addition to the fNIRS testing, a Mood Portraits® study as described in Example 2 was also conducted on a large number of flavoured mouthwash compositions.

For the present invention, the results of the Mood Portraits® study were analysed with regard to a positive happy mood. Specifically, the selection frequency of pictures associated with happiness and the grade of association of the respective pictures with a happy mood (some pictures are very strongly associated with happiness, whereas it is only one association among several equally strong ones for other pictures) were taken into account.

A comparison of several dozen oral care flavour compositions showed that most of them have a very similar effect on positive happy moods; but a few oral care flavour compositions are able to significantly evoke or not evoke a happy mood.

FIG. 2 shows the results for some of the oral care flavour compositions that were tested, namely for Compositions G (according to the invention) and C (comparative example) of Example 3, and of Compositions I through N, which were not previously described in this disclosure.

More precisely, FIG. 2 shows the odds ratio for a happy/uplifted mood, indicating for each oral care flavour composition if it evokes a happy/uplifted mood more or less than the other compositions. The odds ratios are shown as dots. If the 90% confidence interval for the odds ratio for an oral care flavour composition is entirely above the Significance Line of 1.0, then said oral care flavour composition significantly evokes a more positive happy mood and is marked in FIG. 2 by an arrow; if the 90% confidence interval for an oral care flavour composition is entirely below the Significance Line of 1.0, then said oral care flavour composition significantly evokes a less positive happy mood.

Thus, as can be seen from FIG. 2, Composition G, which is an oral care flavour composition according to the present invention, is able to evoke a happy/uplifted mood significantly more than all the other oral care flavour compositions. Compositions C and I through N essentially lie on the Significance Line.

Thus, the Mood Portraits® results confirm that Composition G, which has been found to be happy in the fNIRS study and which also complies with the formulation guidelines of the present invention, significantly evokes more happiness compared to a large majority of other oral care flavour compositions.

### EXAMPLE 6: COMPARISON MOUTHWASH VS. TOOTHPASTE

In order to determine if mouthwash and toothpaste flavours have similar effects on brain activity as determined by fNIRS signals, a protocol was developed where the effects of the same flavour compositions in two different delivery methods (mouthwash and toothpaste) was directly compared.

Fifteen participants took part in the study: each of them attended two separate sessions, at least three hours apart from one another, where they initially were randomly assigned to one of the two conditions (mouthwash or toothpaste). In the second session, they completed the other condition.

The protocol for the two sessions was identical. Upon arrival at the testing location, the experimenter set the fNIRS cap and checked signals quality. Then, once fNIRS recordings started, participants ate a water-based cracker to remove any previous flavour present in their mouth. After eating the cracker, participants were asked to rinse their mouth with water and, after that, use the product evaluated in the session (i.e. to either rinse their mouth with the mouthwash or to brush their teeth with the toothpaste). There was no guidance of time for rinsing/brushing: participants were instructed to use the product as they would normally do at home, to increase the naturalistic element of the study.

The whole sequence of cracker-water-product was repeated four times during each session, with four different flavours, with a minimum of five minutes break between each block to allow for coolants in the products to weaken their effect and allow participants to fully taste the following flavour. Thus, the set-up was identical to that of the fNIRS testing according to Example 1 described above. The four flavours were presented in fully randomised order to the participants.

Brain activity was recorded using fNIRS and analysed during the 30 seconds immediately following each action, once the participant returned to a state of relaxation (i.e. right after finishing eating the cracker, right after spitting/swallowing the water after rinsing and right after spitting the toothpaste/mouthwash).

The analysis was focused on the comparison between the effects on fNIRS signals of the two different product formats (mouthwash vs. toothpaste) in the thirty seconds following their application, by means of ANOVA tests on the four flavours used in the test. Due to the complete lack of references in the scientific literature for studies like this, it was impossible to hypothesise a priori whether it is obvious to expect an increase in fNIRS signals following the application of toothpaste or mouthwash.

Data collected from each participant were pre-processed using a standard fNIRS data pipeline, filtered using a band-pass filter between 0.005 and 0.3 Hz, and then normalised, channel-wise, using the 30 s post-water rinsing interval as a baseline for each application, to make sure the numerical values could be compared across participants in a meaningful way.

To ensure that results were not affected by the specific type of flavour used, the four flavours tested were selected from four different areas of the flavour space. Specifically, flavour 1 was peppermint/herbal/citrus, flavour 2 was spearmint/peppermint, flavour 3 was peppermint/floral, and flavour 4 was spearmint/floral.

The results clearly show that there are no relevant (i.e. statistically significant) differences when the same flavour is applied in mouthwash vs. toothpaste. For example, if the Oxy Hb parameter is considered, there are no significant differences at the full brain, left hemisphere or right hemisphere level (F values, respectively, 1.31, 3.81, 0.21, with all p>0.05). Similarly, if the Deoxy Hb parameter is consider, there are no significant differences at the full brain, left hemisphere or right hemisphere level higher (F values, respectively, 1.46, 0.78, 1.63, with all p>0.05).

To make sure that possible differences in specific flavours were not masked by the group analysis, individual t-test comparisons were run with single flavours (mouthwash vs toothpaste). The results highlighted that, even at the individual flavour level, the two delivery methods for the same flavour composition do not significantly affect brain activity as measured by fNIRS (i.e. there were no relevant differences in the hemodynamic response for mouthwash vs. toothpaste in any of the four flavours).

In particular, the Total Hb values were analysed for all channels and all flavours, and the data showed that, for flavour 1, there was no significant difference in any of the 20 channels analysed; for flavour 2, there was only one statistically significant difference at channel 8; for flavour 3, there was only one statistically significant difference at channel 2; and for flavour 4, there was only one statistically significant difference at channel 5. These statistically significant differences were obtained with a significance threshold of 0.05; however, lowering the threshold to 0.03 makes the flavour 2 difference not significant and further lowering the threshold at 0.01 makes none of the previous differences statistically significant. It is also interesting to notice that, in the three statistically significant cases, mouthwash produced significantly lower brain activity than toothpaste. Therefore, even in the presence of statistically significant differences, there is a clear relationship between the two delivery methods. Overall, out of 80 comparisons in total, 77 (96%) did not show any significant difference between mouthwash and toothpaste in terms of brain activity as measured via fNIRS.

Very similar results were obtained for Oxy Hb and Deoxy Hb, where, out of 80 channels analysed in total on the four flavours, 76 (95%) and 77 (96%), respectively, did not show any statistically significant difference between the two delivery methods and, when differences were present, they were similar to the ones described above (i.e. mouthwash producing significantly lower brain activity than toothpaste, and differences becoming not significant at 0.01 level).

1. A method of assessing the ability of a test oral care flavour ingredient or a test oral care flavour composition to improve the happiness state of a human subject, comprising the steps of:
a) measuring a base happiness state of one or more human test subject(s);
b) providing the test oral care flavour ingredient or the test oral care flavour composition to the human test subject(s) for oral application;
c) measuring a resulting happiness state of the human test subject(s); and
d) determining a difference between the resulting happiness state and the base happiness state for the human test subject(s);
wherein the base happiness state and the resulting happiness state are measured by functional Near Infrared Spectroscopy (fNIRS) of the human test subject(s)' left brain hemisphere, right brain hemisphere, and full brain;
wherein the test oral care flavour ingredient or the test oral care flavour composition is able to improve the happiness state of the human subject if at least one out of the following eight conditions A1 through A8 is met:
A1. Channel 14 shows a statistically significant difference of Total Hb 0-10 seconds after the oral application;
A2. Channel 15 shows a statistically significant difference of Total Hb 0-10 seconds after the oral application;
A3. Channel 18 shows a statistically significant difference of Total Hb 0-10 seconds after the oral application;
A4. Channel 19 shows a statistically significant difference of Total Hb 0-10 seconds after the oral application;
A5. Channel 1 shows a statistically significant difference of Total Hb 0-10 seconds after the oral application;
A6. Channel 3 shows a statistically significant difference of Total Hb 0-10 seconds after the oral application;
A7. Channel 4 shows a statistically significant difference of Total Hb 0-10 seconds after the oral application;
A8. Channel 5 shows a statistically significant difference of Total Hb 0-10 seconds after the oral application;
wherein Total Hb is the amount of total haemoglobin measured, and
wherein Channels 1 to 8 and 11 are located in the left brain hemisphere, Channels 9 and 12 are located on the midline, and Channels 10 and 13 to 20 are located in the right brain hemisphere.

2. The method of claim 1, wherein the test oral care flavour ingredient or the test oral care flavour composition is provided to the human test subject(s) as part of a toothpaste a mouthwash, a breath spray or a dental floss.

3. The method of claim 1, wherein further at least one, more preferably at least two, out of the following nine conditions B1 through B9 is met:
B1. Channel 14 shows a statistically significant difference of Oxy Hb 0-10 seconds after the oral application;
B2. Channel 16 shows a statistically significant difference of Oxy Hb 0-10 seconds after the oral application;
B3. Channel 18 shows a statistically significant difference of Oxy Hb 0-10 seconds after the oral application;
B4. Channel 20 shows a statistically significant difference of Oxy Hb 0-10 seconds after the oral application;
B5. Channel 2 shows a statistically significant difference of Oxy Hb 0-10 seconds after the oral application;
B6. Channel 3 shows a statistically significant difference of Oxy Hb 0-10 seconds after the oral application;
B7. Channel 4 shows a statistically significant difference of Oxy Hb 0-10 seconds after the oral application;
B8. Channel 5 shows a statistically significant difference of Oxy Hb 0-10 seconds after the oral application;
B9. Channel 6 shows a statistically significant difference of Oxy Hb 0-10 seconds after the oral application;
wherein Oxy Hb is the amount of oxygenated haemoglobin measured.

4. The method of claim 1, wherein further at least one, more preferably at least two, out of the following eight conditions C1 through C8 is met:
C1. Channel 16 shows a statistically significant difference of Total Hb 5-10 seconds after the oral application;
C2. Channel 19 shows a statistically significant difference of Total Hb 5-10 seconds after the oral application;
C3. Channel 20 shows a statistically significant difference of Total Hb 5-10 seconds after the oral application;
C4. Channel 1 shows a statistically significant difference of Total Hb 5-10 seconds after the oral application;
C5. Channel 4 shows a statistically significant difference of Total Hb 5-10 seconds after the oral application;
C6. Channel 6 shows a statistically significant difference of Total Hb 5-10 seconds after the oral application;
C7. Channel 9 shows a statistically significant difference of Total Hb 5-10 seconds after the oral application;
C8. Channel 12 shows a statistically significant difference of Total Hb 5-10 seconds after the oral application.

5. A method of creating an oral care flavour composition having effect of improving the happiness of a human subject, comprising the steps of:
(i) creating a test oral care flavour composition;
(ii) assessing the ability of the test oral care flavour composition to improve the happiness state of a human subject according to the method of claim 1, and
(iii) adjusting, if necessary, the test oral care flavour composition by adding and/or removing at least one oral care flavour ingredient and/or increasing and/or reducing the concentration of at least one oral care flavour ingredient until the oral care flavour composition is found to improve the happiness state of the human subject.

6. The method of claim 5, wherein, in step (iii), at least one HMP oral care flavour ingredient is added to the test oral care flavour composition and/or at least one HMR oral care flavour ingredient is added to the test oral care flavour composition and/or at least one HMI oral care flavour ingredient is added to the test oral care flavour composition and/or at least one INV oral care flavour ingredient is removed from the test oral care flavour composition and/or at least one REL oral care flavour ingredient is removed from the test oral care flavour composition and/or the concentration of at least one HMP and/or HMR and/or HMI oral care flavour ingredient is increased and/or the concentration of at least one INV and/or REL oral care flavour ingredient is reduced, wherein
the HMP oral care flavour ingredients are selected from the group consisting of fruity-strawberry ingredients, fruity-raspberry ingredients, fruity-apple ingredients, fruity-banana ingredients, floral-freesia ingredients, floral-lily of the valley ingredients, animalic-butyric ingredients, acetic acid, and mixtures thereof;
the HMR oral care flavour ingredients are selected from the group consisting of fruity-pineapple ingredients, fruity-peach ingredients, herbal-spearmint ingredients, lemon oil, lemon oil reconstitutions, and mixtures thereof;
the HMI oral care flavour ingredients are selected from the group consisting of citrus-orange ingredients, spicy-cinnamon ingredients, floral-jasmine ingredients, cooling ingredients, aromatic-wintergreen ingredients, lime terpenes, lime terpeneless, and mixtures thereof;
the INV oral care flavour ingredients are selected from the group consisting of citrus-lime ingredients (excluding lime terpenes and lime terpeneless), citrus-mandarin ingredients, citrus-grapefruit ingredients, spicy-pepper ingredients, spicy-clove ingredients, herbal-rosemary ingredients, green-grass ingredients, woody-resinous ingredients, herbal-coniferous ingredients, bergamot oils, 3,7-dimethylocta-1,6-dien-3-yl acetate (linalyl acetate), 3,7-dimethylocta-1,6-dien-3-yl 2-methylpropanoate (linalyl iso butyrate), (E)-3,7-dimethylocta-2,6-dienal (citral), lemongrass, Litsea cubeba oils, and mixtures thereof; and
the REL oral care flavour ingredients are selected from the group consisting of sweet-vanilla ingredients, sweet-coconut ingredients, sweet-cooked sugar ingredients, fruity-passionfruit ingredients, herbal-thyme ingredients, sage oils and reconstitutions, clary sage oils and reconstitutions, camomile oils and reconstitutions, lavender oils and reconstitutions, and mixtures thereof.

7. An oral care flavour composition for improving the happiness state of a human subject, the oral care flavour composition comprising:
a) at least one HMP oral care flavour ingredient;
b) at least about 15% by weight in total of HMP and/or HMI and/or HMR oral care flavour ingredients;
c) optionally up to about 85% by weight in total of other oral care flavour ingredients, provided the following conditions are met:
(c1) PEPPERMINTs+INVs<75%
(c2) HMPs+HMIs>RELs
(c3) HMPs+HMRs>INVs
(c4) HMPs/(HMPs+RELs+INVs)≥0.3
wherein
(i) all percentages are based on total weight of the oral care flavour ingredients constituting the oral care flavour composition;
(ii) HMPs indicates the sum of percentages of HMP oral care flavour ingredients; HMIs indicates the sum of percentages of HMI oral care flavour ingredients; HMRs indicates the sum of percentages of HMR oral care flavour ingredients; PEPPERMINTs indicates the sum of percentages of PEPPERMINT oral care flavour ingredients; INVs indicates the sum of percentages of INV oral care flavour ingredients; and RELs indicates the sum of percentages of REL oral care flavour ingredients;
(iii) the symbol ≥ indicates at least equal to;
(iv) the HMP oral care flavour ingredients are selected from the group consisting of fruity-strawberry ingredients, fruity-raspberry ingredients, fruity-apple ingredients, fruity-banana ingredients, floral-freesia ingredients, floral-lily of the valley ingredients, animalic-butyric ingredients, acetic acid, and mixtures thereof;
(v) the HMI oral care flavour ingredients are selected from the group consisting of citrus-orange ingredients, spicy-cinnamon ingredients, floral-jasmine ingredients, cooling ingredients, aromatic-wintergreen ingredients, lime terpenes, lime terpeneless, and mixtures thereof;
(vi) the HMR oral care flavour ingredients are selected from the group consisting of fruity-pineapple ingredients, fruity-peach ingredients, herbal-spearmint ingredients, lemon oil, lemon oil reconstitutions, and mixtures thereof;
(vii) the PEPPERMINT oral care flavour ingredients have a peppermint organoleptic attribute;
(viii) the INV oral care flavour ingredients are selected from the group consisting of citrus-lime ingredients (excluding lime terpenes and lime terpeneless), citrus-mandarin ingredients, citrus-grapefruit ingredients, spicy-pepper ingredients, spicy-clove ingredients, herbal-rosemary ingredients, green-grass ingredients, woody-resinous ingredients, herbal-coniferous ingredients, bergamot oils, 3,7-dimethylocta-1,6-dien-3-yl acetate (linalyl acetate), 3,7-dimethylocta-1,6-dien-3-yl 2-methylpropanoate (linalyl iso butyrate), (E)-3,7-dimethylocta-2,6-dienal (citral), lemongrass, Litsea cubeba oils, and mixtures thereof; and
(ix) the REL oral care flavour ingredients are selected from the group consisting of sweet-vanilla ingredients, sweet-coconut ingredients, sweet-cooked sugar ingredients, fruity-passionfruit ingredients, herbal-thyme ingredients, sage oils and reconstitutions, clary sage oils and reconstitutions, camomile oils and reconstitutions, lavender oils and reconstitutions, and mixtures thereof.

8. The oral care flavour composition of claim 7, comprising at least about 17%, more preferably at least about 20%, by weight in total of HMP and/or HMI and/or HMR oral care flavour ingredients.

9. The oral care flavour composition of claim 7, comprising at least one HMP and/or HMI and/or HMR oral care flavour ingredient selected from one or more of the following groups:
one or more fruity-strawberry ingredients selected from the group consisting of benzyl 3-phenylprop-2-enoate (benzyl cinnamate), ethyl butanoate (ethyl butyrate), ethyl 2-methylpropionate (ethyl isobutyrate), ethyl 3-phenylprop-2-enoate (ethyl cinnamate), methyl 3-phenylprop-2-enoate (methyl cinnamate), ethyl 3-phenyloxirane-2-carboxylate (ethyl phenyl glycidate), ethyl 3-methyl-3-phenyloxirane-2-carboxylate (ethyl methyl phenyl glycidate), 2-phenylethyl butanoate (phenyl ethyl butyrate), benzyl butanoate (benzyl butyrate), ethyl 3-methylbutanoate (ethyl isovalerate), and mixtures thereof;
one or more fruity-raspberry ingredients selected from the group consisting of 4-(4-hydroxyphenyl)butan-2-one (raspberry ketone), methyoxy phenyl butanone, and mixtures thereof,
one or more fruity-apple ingredients selected from the group consisting of ethyl 2-methylbutanoate (ethyl 2-methyl butyrate), ethyl 3-oxobutanoate (ethyl aceto acetate), ethyl 2-methylpentanoate (manzanate), (Z)-hex-3-enyl acetate (cis-3-hexenyl acetate), (E)-2-hexenal (trans-2-hexenal), diethyl propanedioate (diethyl malonate), [(E)-hex-2-enyl] acetate (trans-2-hexenyl acetate), and mixtures thereof;
one or more fruity-banana ingredients selected from the group consisting of pentyl propanoate (amyl propionate), 3-methylbutyl 3-methylbutanoate (iso amyl iso valerate), 6-methylhept-5-en-2-one (methyl heptenone), 3-methylbutyl butanoate (iso amyl butyrate), 3-methylbutyl acetate (iso amyl acetate), pentyl butanoate (amyl butyrate), butyl acetate, 2-methylpropyl acetate (iso butyl acetate), and mixtures thereof;
one or more floral-freesia ingredients selected from the group consisting of 3,7-dimethylocta-1,6-dien-3-ol (linalool), 2-(4-methyl-1-cyclohex-3-enyl)propan-2-yl acetate (terpinyl acetate), 2-(4-methyl-1-cyclohex-3-enyl)propan-2-ol (terpineol alpha), 3,7-dimethylocta-1,6-dien-3-yl (E)-3-phenylprop-2-enoate (linalyl cinnamate), and mixtures thereof;
one or more floral-lily of the valley ingredients selected from the group consisting of 7-hydroxy-3,7-dimethyloctanal (hydroxycitronellal), 2-methyl-3-(4-propan-2-ylphenyl) propanal (cyclamen aldehyde), 3-(1,3-benzodioxol-5-yl)-2-methylpropanal (tropional), 2,6-dimethylhept-5-enal (melonal), and mixtures thereof;
one or more animalic-butyric ingredients selected from the group consisting of butanoic acid (butyric acid), octanoic acid, 2-methylbutanoic acid (2 methyl butyric acid), hexanoic acid (caproic acid), and mixtures thereof.
one or more citrus-orange ingredients selected from the group consisting of orange oil, orange terpenes, and mixtures thereof;
one or more spicy-cinnamon ingredients selected from the group consisting of cassia oils, 3-phenylprop-2-enal (cinnamic aldehyde), 2-methyl-3-phenylprop-2-enal (methyl cinnamic aldehyde), and mixtures thereof;
one or more cooling ingredients selected from the group consisting of N-(4-cyanomethylphenyl) p-menthane carboxamide (Evercool 180), 2-isopropyl-5-methyl-N-(2-(pyridin-2-yl)ethyl)cyclohexane carboxamide (Evercool 190), 3-(5-methyl-2-propan-2-ylcyclohexyl)oxypropane-1,2-diol (Coolact P), (1R,2R,5S)-5-methyl-2-prop-1-en-2-yl-cyclohexan-1-ol (Coolact 10), ethyl 2-[[(1R,2S,5R)-5-methyl-2-propan-2-ylcyclohexane-carbonyl]amino]acetate (WS-5), (1R,2S,5R)—N-(4-methoxyphenyl)-5-methyl-2-(1-methylethyl)cyclohexanecarboxamide (WS-12), N-ethyl-5-methyl-2-propan-2-ylcyclo-hexane-1-carboxamide (WS-3), N,2,3-trimethyl-2-propan-2-ylbutanamide (WS-23), (E)-3-benzo(d)[1,3]dioxol-5-yl)-N,N-diphenylacrylamide (Icool MC6), 2-(p-tolyloxy)-N-(1H-pyrazol-5-yl)-N-((thiophen-2-yl)methyl)acetamide (Freezestorm), [(1R,2S,5R)-5-methyl-2-propan-2-ylcyclohexyl] 2-hydroxypropanoate (Frescolat ML), 2-isopropyl-5-methylcyclohexyloxycarbonyloxy-2-hydroxypropane (Frescolat MGC), 2-hydroxypropyl (5-methyl-2-propan-2-ylcyclohexyl) carbonate (Frescolat MPC), 9-Methyl-6-(1-methylethyl)-1,4-dioxaspiro-[4,5]decan-2-methanol (Frescolat MGA), menthyl lactate, mono-menthyl succinate, menthyl methyl ether, (2E)-3-(1,3-benzodioxol-5-yl)-N,N-diphenylacrylamide (iCool MC 6), 1-methyl-3-hydroxybutyrate, 2-{[5-methyl-2-(propan-2-yl)cyclohexyl]oxy}ethanol (Coolact 5), isomeric mix of 2-(2-hydroxypropan-2-yl)-5-methylcyclohexan-1-ol (Coolact 38D), and mixtures thereof,
one or more aromatic-wintergreen ingredients selected from the group consisting of ethyl 2-hydroxybenzoate (ethyl salicylate), methyl 2-hydroxybenzoate (methyl salicylate), wintergreen oil, and mixtures thereof;
one or more fruity-pineapple ingredients selected from the group consisting of pentyl hexanoate (amyl caproate), prop-2-enyl 3-cyclohexylpropanoate (allyl cyclohexyl propionate), 3-methylbutyl hexanoate (isoamyl caproate), methyl hexanoate, ethyl hexanoate, ethyl heptanoate, 2-phenylethyl 2-methylpropanoate (phenyl ethyl isobutyrate), prop-2-enyl propanoate (allyl propionate), and mixtures thereof;
one or more fruity-peach ingredients selected from the group consisting of 5-heptyldihydrofuran-2(3H)-one (undecalactone gamma), 5-hexyloxolan-2-one (decalactone gamma), 5-octyloxolan-2-one (dodecalatone gamma), 6-heptyloxan-2-one (dodecalatone delta), and mixtures thereof; and
one or more herbal-spearmint ingredients selected from the group consisting of spearmint oils, (1R,5S)-2-methyl-5-prop-1-en-2-ylcyclohex-2-en-1-ol and (1R,5R)-2-methyl-5-prop-1-en-2-ylcyclohex-2-en-1-ol (trans- and cis-carveol) and (5R)-2-methyl-5-prop-1-en-2-ylcyclohex-2-en-1-one (L-Carvone), and mixtures thereof.

10. The oral care flavour composition of claim 7, wherein PEPPERMINTs+INVs<70%, more preferably PEPPERMINTs+INVs<65%, and most preferably PEPPERMINTs+INVs<60%.

11. The oral care flavour composition of claim 7, wherein HMPs/(HMPs+RELs+INVs)≥0.35, more preferably HMPs/(HMPs+RELs+INVs)≥0.40, and most preferably HMPs/(HMPs+RELs+INVs)≥0.45.

12. A consumer product comprising the oral care flavour composition of claim 7.

13. A method of improving the happiness state of a human subject, comprising the step of providing an effective amount of the oral care flavour composition of claim 7 to the human subject.

14. A method of using an oral care flavour ingredient in improving the happiness state of a human subject, wherein the oral care flavour ingredient is selected from the group consisting of:
one or more fruity-strawberry ingredients selected from the group consisting of benzyl 3-phenylprop-2-enoate (benzyl cinnamate), ethyl butanoate (ethyl butyrate), ethyl 2-methylpropionate (ethyl isobutyrate), ethyl 3-phenylprop-2-enoate (ethyl cinnamate), methyl 3-phenylprop-2-enoate (methyl cinnamate), ethyl 3-phenyloxirane-2-carboxylate (ethyl phenyl glycidate), ethyl 3-methyl-3-phenyloxirane-2-carboxylate (ethyl methyl phenyl glycidate), 2-phenylethyl butanoate (phenyl ethyl butyrate), benzyl butanoate (benzyl butyrate), ethyl 3-methylbutanoate (ethyl isovalerate), and mixtures thereof;
one or more fruity-raspberry ingredients selected from the group consisting of 4-(4-hydroxyphenyl)butan-2-one (raspberry ketone), methyoxy phenyl butanone, and mixtures thereof,
one or more fruity-apple ingredients selected from the group consisting of ethyl 2-methylbutanoate (ethyl 2-methyl butyrate), ethyl 3-oxobutanoate (ethyl aceto acetate), ethyl 2-methylpentanoate (manzanate), (Z)-hex-3-enyl acetate (cis-3-hexenyl acetate), (E)-2-hexenal (trans-2-hexenal), diethyl propanedioate (diethyl malonate), [(E)-hex-2-enyl] acetate (trans-2-hexenyl acetate), and mixtures thereof;
one or more fruity-banana ingredients selected from the group consisting of pentyl propanoate (amyl propionate), 3-methylbutyl 3-methylbutanoate (iso amyl iso valerate), 6-methylhept-5-en-2-one (methyl heptenone), 3-methylbutyl butanoate (iso amyl butyrate), 3-methylbutyl acetate (iso amyl acetate), pentyl butanoate (amyl butyrate), butyl acetate, 2-methylpropyl acetate (iso butyl acetate), and mixtures thereof;
one or more floral-freesia ingredients selected from the group consisting of 3,7-dimethylocta-1,6-dien-3-ol (linalool), 2-(4-methyl-1-cyclohex-3-enyl)propan-2-yl acetate (terpinyl acetate), 2-(4-methyl-1-cyclohex-3-enyl)propan-2-ol (terpineol alpha), 3,7-dimethylocta-1,6-dien-3-yl (E)-3-phenylprop-2-enoate (linalyl cinnamate), and mixtures thereof;
one or more floral-lily of the valley ingredients selected from the group consisting of 7-hydroxy-3,7-dimethyloctanal (hydroxycitronellal), 2-methyl-3-(4-propan-2-ylphenyl) propanal (cyclamen aldehyde), 3-(1,3-benzodioxol-5-yl)-2-methylpropanal (tropional), 2,6-dimethylhept-5-enal (melonal), and mixtures thereof;
one or more animalic-butyric ingredients selected from the group consisting of butanoic acid (butyric acid), octanoic acid, 2-methylbutanoic acid (2 methyl butyric acid), hexanoic acid (caproic acid), and mixtures thereof.
one or more citrus-orange ingredients selected from the group consisting of orange oil, orange terpenes, and mixtures thereof;
one or more spicy-cinnamon ingredients selected from the group consisting of cassia oils, 3-phenylprop-2-enal (cinnamic aldehyde), 2-methyl-3-phenylprop-2-enal (methyl cinnamic aldehyde), and mixtures thereof;
one or more cooling ingredients selected from the group consisting of N-(4-cyanomethylphenyl) p-menthane carboxamide (Evercool 180), 2-isopropyl-5-methyl-N-(2-(pyridin-2-yl)ethyl)cyclohexane carboxamide (Evercool 190), 3-(5-methyl-2-propan-2-ylcyclohexyl)oxypropane-1,2-diol (Coolact P), (1R,2R,5S)-5-methyl-2-prop-1-en-2-yl-cyclohexan-1-ol (Coolact 10), ethyl 2-[[(1R,2S,5R)-5-methyl-2-propan-2-ylcyclo-hexanecarbonyl]amino]acetate (WS-5), (1R,2S,5R)—N-(4-methoxyphenyl)-5-methyl-2-(1-methylethyl)cyclohexanecarboxamide (WS-12), N-ethyl-5-methyl-2-propan-2-yl-cyclohexane-1-carboxamide (WS-3), N,2,3-trimethyl-2-propan-2-ylbutanamide (WS-23), (E)-3-benzo(d)[1,3]dioxol-5-yl)-N,N-diphenylacrylamide (Icool MC6), 2-(p-tolyloxy)-N-(1H-pyrazol-5-yl)-N-((thiophen-2-yl)methyl)acetamide (Freezestorm), [(1R,2S,5R)-5-methyl-2-propan-2-ylcyclohexyl] 2-hydroxypropanoate (Frescolat ML), 2-isopropyl-5-methylcyclohexyloxycarbonyloxy-2-hydroxypropane (Frescolat MGC), 2-hydroxypropyl (5-methyl-2-propan-2-ylcyclohexyl) carbonate (Frescolat MPC), 9-Methyl-6-(1-methyl-ethyl)-1,4-dioxaspiro-[4,5]decan-2-methanol (Frescolat MGA), menthyl lactate, mono-menthyl succinate, menthyl methyl ether, (2E)-3-(1,3-benzodioxol-5-yl)-N,N-diphenyl-acrylamide (iCool MC 6), 1-methyl-3-hydroxybutyrate, 2-{[5-methyl-2-(propan-2-yl)cyclohexyl]oxy}ethanol (Coolact 5), isomeric mix of 2-(2-hydroxypropan-2-yl)-5-methylcyclohexan-1-ol (Coolact 38D), and mixtures thereof,
one or more aromatic-wintergreen ingredients selected from the group consisting of ethyl 2-hydroxybenzoate (ethyl salicylate), methyl 2-hydroxybenzoate (methyl salicylate), wintergreen oil, and mixtures thereof:
one or more fruity-pineapple ingredients selected from the group consisting of pentyl hexanoate (amyl caproate), prop-2-enyl 3-cyclohexylpropanoate (allyl cyclohexyl propionate), 3-methylbutyl hexanoate (isoamyl caproate), methyl hexanoate, ethyl hexanoate, ethyl heptanoate, 2-phenylethyl 2-methylpropanoate (phenyl ethyl isobutyrate), prop-2-enyl propanoate (allyl propionate), and mixtures thereof;
one or more fruity-peach ingredients selected from the group consisting of 5-heptyldihydrofuran-2(3H)-one (undecalactone gamma), 5-hexyloxolan-2-one (decalactone gamma), 5-octyloxolan-2-one (dodecalatone gamma), 6-heptyloxan-2-one (dodecalatone delta), and mixtures thereof; and
one or more herbal-spearmint ingredients selected from the group consisting of spearmint oils, (1R,5S)-2-methyl-5-prop-1-en-2-ylcyclohex-2-en-1-ol and (1R,5R)-2-methyl-5-prop-1-en-2-ylcyclohex-2-en-1-ol (trans- and cis-carveol) and (5R)-2-methyl-5-prop-1-en-2-ylcyclohex-2-en-1-one (L-Carvone), and mixtures thereof.
